(19) United States
(12) Patent Application Publication
(10) Pub. No.: US 2005/0164210 A1
(43) Pub. Date: Jul. 28, 2005

(54) Regulated polymerase III expression systems and related methods

(76) Inventors: Vivek Mittal, Syosset, NY (US); Sunita Gupta, Hauppauge, NY (US); Gregory J. Hannon, Huntington, NY (US); Patrick J. Paddison, Oyster Bay, NY (US); Eric Julien, Limoges (FR); Winship Herr, St-Sulpice (CH)

Correspondence Address: FISH & NEAVE IP GROUP;ROPES & GRAY LLP, ONE INTERNATIONAL PLACE, BOSTON, MA, 02110-2624, US

(21) Appl. No.: 10/763,479

(22) Filed: Jan. 23, 2004

(51) Int. Cl. C12Q 1/68; A01K 67/00; C07H 21/04; C12N 9/22

(52) U.S. Cl. 435/6; 435/69.1; 435/199; 435/320.1; 435/325; 536/23.2; 800/8

(57) ABSTRACT

The invention provides, among other things, regulated polymerase III expression systems and related compositions. The invention provides, in part, expression systems in which the expression is inducible, and systems which express inhibitory RNA molecules, such as hairpin RNA molecules. The invention also provides related methods, such as methods of inhibiting expression of a gene.

### GOVERNMENT SUPPORT

Work described herein was funded, in whole or in part, by grants CA13106 and CA87497 from NCI and a grant R01-GM62534 from NIH. The United States Government has certain rights in the invention.

### BACKGROUND OF THE INVENTION

The ability to vary expression levels of an endogenous gene product at will and to monitor effects on cells or whole animals can provide useful insights into its biological role. RNAi-mediated gene silencing has emerged as powerful approach to regulate levels of an endogenous protein within its physiological limits.

RNAi is a process of sequence-specific post-transcriptional gene silencing mediated by double stranded RNA and is an effective genetic approach to analyze gene function in many organisms (1, 2). The endogenous mediators of sequence-specific mRNA degradation are 21- and 22-nucleotide siRNAs generated from longer dsRNAs by the ribonuclease III activity of the evolutionary conserved dicer enzyme (3, 4). Gene-specific long dsRNAs have been successfully used in worms and flies for RNAi mediated gene silencing (5). However, in mammalian cells dsRNA longer then 30 base pairs trigger the antiviral/interferon pathways that results in global shut-down of protein synthesis (6, 7). Recently it was demonstrated that RNAi-mediated gene silencing can be obtained in cultured mammalian cells by delivery of chemically synthesized short (<30 nt) double-stranded siRNA molecules (3) or by endogenous expression of short hairpin RNAs (shRNAs) bearing a fold back stem-loop structure (8-12).

Plasmid- and viral vector-based constitutive expression of shRNAs by RNA polymerase III (pol III) U6 and H1 snRNA promoters (U6 or H1) often result in stable and efficient suppression of target genes (9, 10, 13). However, the inability to adjust levels of suppression has imposed limitations in the analysis of genes essential for viability, cell survival, cell-cycle regulation and development. Besides, gross suppression of a gene over longer periods of time may result in non-physiological responses. This problem can be circumvented by expressing RNAi molecules under regulated promoter. For example, shRNA constructs can be expressed under tissue specific promoters in an organism or can be expressed using inducible promoters in mammalian cells. The two most widely used inducible mammalian systems use tetracycline- or ecdysone-responsive transcriptional elements (14,15). The chief drawback of the tetracycline-inducible system is a relatively high background of expression in the uninduced state in certain cell lines (15, 16). The ecdysone-inducible system is tightly regulated (15, 17), with no expression in the uninduced state and a rapid inductive response (15), and importantly, the components of the inducible system is inert with rapid clearance kinetics and therefore, does not affect mammalian physiology.

Therefore, a need remains to develop inducible polymerase III expression systems for the expression of transgenes, and in particular shRNA or siRNAs for regulated inhibition of genes. Furthermore, a need remains for systems which reversibly reduce gene expression.

### SUMMARY OF THE INVENTION

In some aspects, the invention provides a system for the regulated expression of an RNA molecule. In some aspects, the invention provides systems for expressing double-stranded or hairpin RNA molecules transcribed by RNA polymerase III under inducible, tissue specific, developmental, temporal or other modes of regulation. The system provided by the invention may be applied to any eukaryotic cell, and in particular, to mammalian cells. In some preferred aspects, the RNA molecule inhibits gene expression of a gene, allowing for the regulated inhibition of gene expression in a cell or in an organism, particularly of genes which are essential for cell viability or whose function may overlap with that of other genes which may compensate for a loss-of-function in a gene of interest over time.

Some aspects of the invention provide kits, compositions, nucleic acids, cells, and organisms which comprise components of the regulated RNA-expression systems described herein, such as components of the regulated polymerase III expression systems. In other aspects, the invention provides methods of using the regulated RNA-expression systems described herein. Such methods include, but are not limited to, methods of reducing the expression level or activity of a gene, methods of determining the effects of silencing expression of a gene, methods of identifying genes whose knockdown effects a desired phenotype, and methods of identifying gene targets for agents and/or therapeutics, such as drugs used to treat subjects afflicted or at risk of being afflicted with a disorder.

One aspect of the invention provides a regulated polymerase III expression system, comprising (a) a regulated promoter operably linked to a first element encoding a transcription factor; and (b) a recombinant polymerase III promoter regulated by the transcription factor, wherein binding of the transcription factor to (i) the polymerase III promoter or to (ii) a binding site operably linked to the polymerase III promoter increases transcription from the recombinant polymerase III promoter. Another aspect provides a regulated polymerase III expression system, comprising (a) a first nucleic acid segment comprising a regulated promoter operably linked to a first element encoding a transcription factor; and (b) a second nucleic acid segment comprising a recombinant polymerase III promoter regulated by the transcription factor, wherein the transcription factor increases transcription from the recombinant polymerase III promoter. In some embodiments, binding of the transcription factor to (i) the polymerase III promoter or to (ii) at least one binding site operably linked to the polymerase III promoter increases transcription from the recombinant polymerase III promoter.

In some embodiments, the regulated promoter is an inducible promoter, such as a ecdysone-inducible promoter. In yet other embodiments, the regulated polymerase III expression systems drives the transcription of a short hairpin RNA (shRNA), such as shRNAs which silence genes essential for cell survival, viability, cell-cycle regulation and development.

Another aspect of the invention provides nucleic acids, such as plasmids and viral vectors, cells or animals, which comprise components of the regulated polymerase III expression system. In some aspects these nucleic acid, cells or animals are provided as kits.

The invention also provides a method of reducing expression of a gene in a cell, the method comprising (a) providing a cell comprising (i) a regulated promoter operably linked to a first element encoding a transcription factor; and (ii) a recombinant polymerase III promoter regulated by the transcription factor and operably linked a coding sequence for an RNA molecule, wherein expression of the RNA molecule reduces expression of a gene; and (b) contacting the cell with an inducer, wherein the inducer promotes transcription of the RNA molecule from the recombinant polymerase III promoter, thereby reducing expression of the gene in the cell.

Furthermore, the invention provides a method of determining the effects of reducing expression of a gene. In a specific embodiment, the invention provides a method of determining the effects of reducing expression of a gene, comprising (a) providing a cell comprising (i) a regulated promoter operably linked to a first element encoding a transcription factor; and (ii) a recombinant polymerase III promoter regulated by the transcription factor and operably linked a coding sequence for an RNA molecule, wherein expression of the RNA molecule reduces expression of the gene; (b) subjecting the cell to a condition which promotes transcription of the RNA molecule from the recombinant polymerase III promoter; and (c) determining the phenotype of the cell, thereby determining the effects of reducing expression of a gene.

The invention additionally provides methods of determining the effects of silencing expression of a gene in an organism. One specific embodiment provides a method of determining the effects of silencing expression of a gene in an organism comprising (a) providing an organism wherein at least a cell in the organism comprises (i) a regulated promoter operably linked to a first element encoding a transcription factor; and (ii) a recombinant polymerase III promoter regulated by the transcription factor and operably linked a coding sequence for an RNA molecule, wherein expression of the RNA molecule reduces expression of the gene; (b) subjecting the organism to conditions which promote transcription of the RNA molecule from the recombinant polymerase III promoter in at least one cell; and (c) determining the phenotype of at least one cell in the organism; thereby determining the effects of silencing expression of a gene in an organism.

### BRIEF DESCRIPTION OF THE DRAWINGS

FIG. 1 illustrates the design of retroviral vectors for ecdysone-inducible synthesis of shRNA and experimental validation in mammalian cells. (A) Vector descriptions. pVgEcR-MP, retroviral vector for constitutive expression of a modified Drosophila ecdysone receptor (containing the VP16 transactivating domain) from the LTR and marked with puromycin resistance gene; pRXR-MN, retroviral vector for constitutive expression of the RXR from the LTR and marked with the neomycin (G418)-resistance gene; pEind-RNAi, a self-inactivating retroviral ecdysone-inducible vector marked with hygromycin resistance gene. E/GRE: hybrid ecdysone response element; Hsmin, minimal heat shock promoter; IRES, Internal ribosomal entry site; EGFP; enhanced green fluorescent protein; PUR, puromycin; NEO, G418 resistance gene. LTR; long terminal repeat; Tkp, enhancerless thymidine kinase promoter; PGK, phospho-glucokinase promoter; ccdB, Gateway system cassette. (B) Microscopic analysis of a representative cell line to show GFP⁺ cells in the absence of murA (−murA) and presence of 0.5 μM murA (+murA) at 72 h post-induction. (C) FACS analysis to show GFP⁺ cells in the absence (−murA) and presence (+murA) of 5 μM murA at 72 h post-induction. (D) Western blot analysis showing expression of EGFP and GAL4-Oct-2^{Q}(Q→A) in uninduced cells (lane 1) and cells induced with 5 μM MurA (lane 2) for 72 h. (E). Northern blot analysis showing inducible expression of p53-specific siRNAs in cells treated with 5 μM murA for 72 h, by probing with ³²P-labeled p53 sense strand. 18S RNA served as an internal control to show equal loading.

FIG. 2 demonstrates the stable and efficient RNAi-mediated inducible suppression of human p53 gene in U87MG cells. (A) Dose response of ecdysone-inducible RNAi. Stable cell lines carrying p53 specific shRNA (p53-SP) and non-specific shRNA (NON-SP) were induced with 0.0 (lane 1), 0.5 (lane 2), 2.0 (lane 3), 3.0 (lane 4) and 5.0 (lane 5) μM murA. Whole cell extracts were prepared after 72 h and analyzed by Western blotting for p53, p21 and β-tubulin (control). Fold-reduction in p53 protein level is 30% (lane 2), 64% (lane 3), 90% (lane 4) and >95% (lane 5) relative to uninduced sample (lane 1). (B) Time-course of ecdysone-inducible RNAi. Stable cells carrying shRNA for p53 were induced with 5 μM murA and analyzed for p53 levels at indicated time points and fold-reduction of p53 protein level is 60% (+murA at 48 h), where as (+murA at 96 h) is >95%. Fold-reduction of protein level was based on densitometric measurement. (C) Inducible suppression of p53 at a single cell level by immunofluorescence showing silencing of p53 gene by an inducible p53 shRNA in the presence of 5 μM murA (upper panel, bottom) but not in the absence of murA (upper panel, top), 72 h post-induction. Staining with p53-specific antibody (red), α-tubulin (green). DAPI was used to stain nuclei (blue). (D) Cell cycle analysis of γ-irradiated U87MG cells carrying inducible p53 shRNA by FACS. Stable cell lines carrying p53 shRNA were either uninduced (−murA) or induced (+murA) with 5 μM murA for 72 h and subjected to 20 Gy of γ-irradiation. After 24 h FACS analysis showing G2/M arrest (upper panel, right). IR, irradiation.

FIG. 3 shows RNAi-mediated inducible gene suppression is reversible. (A) Western blot analysis showing 93% fold-reduction in p53 protein levels in cells induced with 5 μM murA (+murA) relative to uninduced (−murA) at 72 h. Following murA removal the fold-recovery in p53 levels is 20% (48 h) and >90% (96 h). (B) Phase contrast microscopy showing morphology of U87MG cells either uninduced (−murA) or induced with 5 μM murA (+murA) for 72 h and at 48 and 96 h following murA removal.

FIG. 4 shows inducible suppression of MyoD gene expression in a murine endothelial cells. (A) Cells stably and inducibly expressing MyoD shRNA (MyoD-SP) and non-specific shRNA (NON-SP) were treated with 5 μM murA. Whole cell extracts were prepared at 72 h post-induction and analyzed by Western blot for MyoD and β-tubulin. Fold-reduction of protein level is >95% for MyoD (MyoD-SP) relative to non-specific shRNA. (B) Stable cells carrying MyoD specific shRNA (MyoD-SP) and non-specific shRNA (NON-SP) were induced with 0.0 (lane 1), 0.5 (lane 2), 1.0 (lane 3), 3.0 (lane 4) and 5.0 (lane 5) μM murA and subjected to Western blot for MyoD and β-tubulin. Fold-reduction of MyoD protein level for shRNA (MyoD-SP) is 5% (lane 2), 25% (lane 3), 72% (lane 4) and >95% (lane 5) relative to uninduced sample (lane 1). Fold-reduction in MyoD was normalized against β-tubulin for each band. shRNA used for the suppression of MyoD gene expression was identified in a high throughput screen (19).

FIG. 5 shows the derivation of inducible cell lines. Cells were transduced at high efficiency with receptor viruses (pRXR-MN and pVgEcR-MP) and cells resistant to puromycin and G418 were isolated, expanded and used for transduction with a third virus (pEind-RNAi), carrying a shRNA and further selected in hygromycin. Next, the cells were induced with a very low dose of murA (1-3 μM) and the top 3% GFP⁺ cells were sorted by FACS and expanded. Cells expressing GFP in the absence of the inducer were considered as “leaky” (about 2% of the population), were removed by flow cytometry and expanded for further analysis. Sorted cells were induced with 0.5 μM murA for 72 h and visualized under a fluorescent microscope.

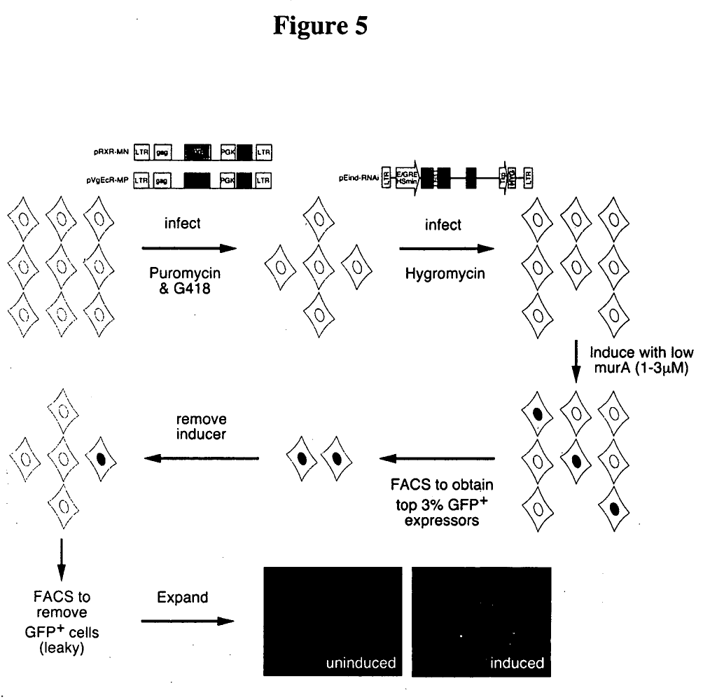

### DETAILED DESCRIPTION OF THE INVENTION

I. Overview

In certain aspects, the invention provides methods, compositions, nucleic acids and systems for the regulated expression of transgenes or other genetic elements from a polymerase III promoter. In some aspects, the invention provides methods and nucleic acids for expression of inhibitory hairpin RNAs from a regulated polymerase III promoter. In other aspects, the invention provides methods of reducing the gene expression of a gene using regulated expression of inhibitory RNA molecules in a cell or in an organism, and further provides methods of determining the effects or phenotype of reducing gene expression of a gene in a cell or in an organism.

RNA interference (RNAi) is a powerful genetic approach for efficiently silencing target genes. The existing methods of gene suppression by the constitutive expression of short hairpin RNAs (shRNAs) allow analysis of the consequences of stably silencing genes, but limit the analysis of genes essential for cell survival, cell-cycle regulation and development.

To overcome this limitation, the invention described herein provides, in certain aspects, a regulated polymerase III expression system and related methods for silencing genes with inhibitory RNAs in a regulated manner. The invention is based, in part, on a two component system. The first component comprises a transcription factor encoded by a first element, wherein transcription of the first element is under the control of a regulated promoter. In some alternate embodiments, the first element encodes a transcriptional repressor. The regulated promoter may be an inducible promoter, such as a promoter which is transcriptionally active in the presence of an inducer, such as an ecdysone or an analog or mimic thereof. The regulated promoter may also be a promoter which is not constitutively active, such as a promoter which is developmentally regulated or cell-cycle regulated. In specific embodiments, the regulated promoters of the present invention comprise those which become active or inactive when the cell in which they reside is contacted with an extracellular molecule, such as a growth factor, a hormone, a cytokine, a chemokine, a transmembrane protein, or an extracellular matrix protein. The regulated promoter may also comprise a tissue-specific promoter. In some embodiments, the regulated promoter comprises both a promoter, which may be a basal, regulated, or inducible promoter, and a response element, or a binding site for a regulatory, operably-linked to the promoter, such that a regulatory protein binding to the response element regulates transcription from the regulated promoter. Many transcription factors which may serve as regulatory proteins and the sequence of their respective binding sites are well-known to one skilled in the art.

The second component comprises a recombinant polymerase III promoter under the control of the transcription factor or of the transcriptional repressor. In specific embodiments, the polymerase III promoter comprises, or is operably-linked to, at least one binding site for the transcription factor, such that binding of the transcription factor to the binding site promotes transcription from the polymerase III promoter. In preferred embodiments, the polymerase III promoter directs transcription of a transgene, such as a transgene encoding a short hairpin RNA or an siRNA. In some embodiments of the regulated polymerase III transcription system and related methods, the silencing of a gene by an inhibitory RNA molecule whose expression is under the control of the regulated polymerase III promoter, is reversible. Examples of such reversible systems are provided in the experimental section.

The invention also provides nucleic acids comprising the two components of the regulated polymerase transcription system, such as one nucleic acid comprising both components or two nucleic acids each comprising one component. The invention further provides cells comprising any of these nucleic acids.

The regulated polymerase III transcription system may be used to knock-down the expression of a gene by selecting a suitable inhibitory short hairpin RNA or siRNA. Accordingly, the invention provides methods for silencing or reducing gene expression of a gene, and methods of determining the effects of silencing expression of a gene. The methods described herein may be applied to a cell in vitro or vivo, or to a cell or cells in an organism. When used in an organism, the regulated polymerase III transcription system may be used to silence a gene in a specific cell type, in multiple cell types or in all cell types. The system may also be used to silence a gene at desired developmental stages or by using an inducer in a spatially or temporally restricted matter. In some embodiments, the systems provided herein may be used to silence a gene reversibly.

Contemporaneous reports describe polymerase III promoters regulated by tetracycline-based suppressors (Wiznerowicz M, Trono D. J. Virol. 2003 August; 77(16): 8957-61; Czauderna F et al. 2003. Nucleic Acids Res. 31(21): e127; van de Wetering et al. 2003. EMBO Rep.; 4(6): 609-15; Ohkawa, J. and Taira, K. 2000. Human Gene Therapy 11(4): 577-685). However, tetracycline-based expression systems show high expression in some cell lines, may be toxic to cells and may show variable results when introduced into transgenic animals (Saez et al., 1997. Current Opinion in Biotechnology 8: 608-616).

In one aspect, the invention relates to an inducible U6 promoter for synthesis of shRNAs in both human and murine cells. Cells containing stably integrated shRNA expression constructs, described herein, demonstrate stringent dosage- and time-dependent kinetics of induction with undetectable background expression in the absence of the inducer ecdysone.

Inducible suppression of human p53 in glioblastoma cells using the expression systems described herein result in striking morphological changes and defects in cell-cycle arrest caused by DNA damage, as expected. Remarkably, the inducibility is reversible following withdrawal of the inducer as observed by reappearance of the protein and a restoration of the original cell phenotype. Inducible and reversible regulation of RNAi has broad applications in the areas of mammalian genetics and molecular therapeutics.

In specific embodiments, the invention provides, to facilitate stable and inducible suppression of any gene, an ecdysone-inducible synthesis of short hairpin RNAs (shRNAs) under the control of a modified polymerase III specific U6 promoter. Using a retroviral delivery and fluorescence-activated cell sorting (FACS) analysis of enhanced green fluorescence protein (EGFP) positive cells, applicants have shown that stable cell lines comprising embodiments of the regulated polymerase expression system described herein are rapidly and efficiently established using either murine or human cells, thus alleviating the labor-intensive isolation and analysis of multiple independent clones. Some embodiments of the expression systems described herein provide RNAi inducibility with stringent dose and time-dependent kinetics of induction with undetectable background expression in the absence of the inducer in cells. For instance, in the Exemplification, Applicants inducibly expressed shRNAs targeting the human tumor suppressor p53 gene in the human glioblastoma cell line and MyoD in the murine endothelial cell line. Dose and time-dependent suppression of p53 gene expression was associated with changes in cell morphology and concomitant reduction in its downstream target p21. Furthermore, the suppression was specific as it could override p53 dependent cell cycle arrest caused by γ-irradiation. Remarkably, withdrawal of the inducer completely reversed the phenotype as indicated by reappearance of the protein, a restoration of original morphology, and a gain in the ability to undergo p53-mediated cell cycle arrest in response to γ-irradiation. Inducible regulation of RNAi with reversible properties, using the expression systems described herein, has broad utility in the areas of mammalian genetics and molecular therapeutics.

II. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

A “patient” or “subject” to be treated by the method of the invention can mean either a human or non-human animal, preferably a mammal.

The term “encoding” comprises an RNA product resulting from transcription of a DNA molecule, a protein resulting from the translation of an RNA molecule, or a protein resulting from the transcription of a DNA molecule and the subsequent translation of the RNA product.

The term “expression vector” and equivalent terms are used herein to mean a vector which is capable of inducing the expression of DNA that has been cloned into it after transformation into a host cell. The cloned DNA is usually placed under the control of (i.e., operably linked to) certain regulatory sequences such a promoters or enhancers. Promoters sequences maybe constitutive, inducible or repressible.

The term “substantially pure” or “purified” are used herein to mean that the desired product is essentially free from contaminating cellular components. Containments may include, but are not limited to, proteins, carbohydrates and lipids. One method for determining the purity of a protein or nucleic acid is by electrophoresis in a matrix such as polyacrylamide or agarose. Purity is evidence by the appearance of a single band after staining.

Any prokaryotic or eukaryotic cell that is the recipient of a vector is the host for that vector. The term encompasses prokaryotic or eukaryotic cells that have been engineered to incorporated a gene in their genome. Cells that can serve as hosts are well known in the art as are techniques for cellular transformation (see e.g., Molecular Cloning: A Laboratory Manual, 3rd Ed., ed. by Sambrook and Russell (Cold Spring Harbor Laboratory Press: 2001).

The term “promoter” is used herein to mean a DNA sequence that initiates the transcription of a gene. Promoters are typically found 5′ to the gene and located proximal to the start codon. If a promoter is of the inducible type, then the rate of transcription increases in response to an inducer. Promoters may be operably linked to DNA binding elements that serve as binding sites for transcriptional regulators. The term “mammalian promoter” is used herein to mean promoters that are active in mammalian cells. Similarly, “prokaryotic promoter’ refers to promoters active in prokaryotic cells.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

The term “recombinant” is used herein to mean any nucleic acid comprising sequences which are not adjacent in nature. A recombinant nucleic acid may be generated in vitro, for example by using the methods of molecular biology, or in vivo, for example by insertion of a nucleic acid at a novel chromosomal location by homologous or non-homologous recombination.

The term “operably linked” is used herein to mean molecular elements that are positioned in such a manner that enables them to carry out their normal functions. For example, a gene is operably linked to a promoter when its transcription is under the control of the promoter and, if the gene encodes a protein, such transcription produces the protein normally encoded by the gene. For example, a binding site for a transcriptional regulator is said to be operably linked to a promoter when transcription from the promoter is regulated by protein(s) binding to the binding site. Likewise, two protein domains are said to be operably linked in a protein when both domains are able to perform their normal functions.

The term “gene” is used herein to mean a nucleic acid sequence that undergoes transcription as the result of promoter activity. A gene may code for a particular protein or, alternatively, code for an RNA sequence, such as a hairpin RNA, that is of interest in itself, e.g. because it acts as an antisense inhibitor.

III. Regulated Polymerase III Expression System

One aspect of the invention provides a regulated polymerase III expression system, comprising (a) a regulated promoter operably linked to a first element encoding a transcription factor; and (b) a recombinant polymerase III promoter regulated by the transcription factor, wherein the transcription factor increases transcription from the recombinant polymerase III promoter. The two components of the regulated polymerase expression system may reside in the same nucleic acid or may reside in two or more nucleic acids. Accordingly, in one embodiment, the (a) the regulated promoter and the first element; and (b) the recombinant polymerase III promoter of the regulated polymerase III expression system reside in the same nucleic acid, while in another embodiment they reside in separate nucleic acids.

Another aspect of the invention provides a regulated polymerase III expression system, comprising (a) a first nucleic acid segment comprising a regulated promoter operably linked to a first element encoding a transcription factor; and (b) a second nucleic acid segment comprising a recombinant polymerase III promoter regulated by the transcription factor, wherein the transcription factor increases transcription from the recombinant polymerase III promoter. The first nucleic acid segment and the second nucleic acid segment may reside on the same nucleic acid or they may reside is separate nucleic acids. For example, in one embodiment, a viral vector comprises both the first and second nucleic acid segments. Such viral vectors may be used to introduced the expression system into a cell, such as a mammalian or a plant cell.

In one embodiment of the regulated polymerase III expression systems described herein, the regulated promoter is an inducible promoter. In one embodiment, the regulated promoter comprises, or is operably-linked to, an ecdysone inducible element or to a Tet responsive element. Ecdosyne inducible elements are described, for example, in WO 97/38117 and U.S. Patent Publication 2002/0177564. In one specific embodiment, transcription from the inducible promoter is increased by an ecdysone, an analog or mimic thereof, such as muristerone A. In one embodiment, the inducer is a caged compound, such as that described in Lin, et al. (2002) Chem Biol 9, 1347-53. The use of caged compounds allows release of the inducer in a tissue, spatial and time-dependent manner. The regulated promoter of the regulated polymerase III expression systems described herein is not limited to inducible promoters whose transcription is activated by a chemical inducer. In some embodiments, the regulated promoter is activated by temperature, such as heat shock promoters.

In other embodiments, the regulated promoter is a promoter whose transcription is regulated by the signaling pathways initiated by extracellular signaling molecules, such as growth factors, hormones, cytokines, chemokines, a transmembrane proteins, or extracellular matrix proteins. In some embodiments, the extracellular signaling molecule is amphiregulin, angiopoietin 1 to angiopoietin 4, AP03 ligand, BMP-2 to BIVIP-15, BDNF, betacellulin, cardiotrophin-1, CD27 ligand, CD30 ligand, CD40 ligand, CNTF, EGF, epiregulin, erythropoietin, Fas ligand, FGF-1 to FGF-19, Flt-3 ligand, G-CSF, GDF-1, GDF-3, GDF-8 to GDF-10, GITR ligand, GM-CSF, heparin binding-EGF, hepatocyte growth factor, IFN-γ, IFN-α, IFN-β, IGF-1, IGF-11, inhibin A, inhibin B, IL-1α, IL-1β, IL-2α, IL-7, IL-9 to IL-11, IL-12, p35, IL-12, p401, IL-13 to IL-19, leptin, LIF, LIGHT, LT-P, lymphotactin, M-CSF, midkine, MIS, macrophage stimulating protein, neuregulin, NGF, NT-3, NT-4, NT-6, oncostatin M, OX40 ligand, PDGF-A, PDGF-B, placenta growth factor, pleiotrophin, SMDF, SCF, TALL-1, TALL-2, TGF-α, EPO, TNFα, TNF-β, TRAIL, TRANCE, VEGF-A, VEGF-B, VEGF-C, VEGF-D, and VEGI.

In other embodiments, the promoters are specifically active or inactive under specific cellular conditions or stresses, including DNA damage, apoptosis, cell division, hypoxia or differentiation. In specific embodiments, the regulated promoter is a cell-cycle regulated promoter, or the regulated promoter comprises an enhancer which confers cell-cycle regulated expression. A cell-cycle regulated promoter is a promoter whose transcriptional state is upregulated or downregulated during at least one stage of the cell cycle. In one embodiment, the regulated promoter of the regulated polymerase III expression system described herein is a temporally-regulated promoter or a developmental-promoter. In another embodiment, the regulated promoter is a tissue specific promoter. Tissue specific promoters include, but are not limited to, cardiac muscle-specific promoters, skeletal muscle-specific promoters, endothelial cell-specific promoters, neuron-specific promoters, glia-specific promoters, retina-specific promoters, kidney-specific promoters, liver-specific promoters, lung-specific promoters, lymphocyte-specific promoters, myeloid-specific promoters, and tumor-specific promoters.

In some embodiments of the regulated polymerase III expression systems described herein, the regulated promoter comprises a developmentally-regulated promoter. Such embodiments allow the expression of a transgene operably linked to the polymerase III promoter to be expressed only at specific developmental stages such as during gastrulation of an embryo, during cardiac myogenesis, or during puberty. A regulated promoter which is a developmentally-regulated promoter allows the inhibition of a gene expression at specific developmental stages when a transgene encoding an inhibitory RNA is operably linked to the polymerase III promoter.

In other embodiments of the regulated polymerase III expression systems described herein, the regulated promoter comprises the promoter for any of the following mammalian genes: adenine nucleotide transporter-2, albumin, aldehyde dehydrogenase-3, B29/ig-P, cardiac actins or myosin heavy chains, CD95/Fas/AP01, crystallins, dopamine, P-hydroxylase, elastase, endothelins, enolases, erythropoietin, a-fetoprotein, globins, glucocorticoid receptor, glutathione P transferase, growth hormone, heat shock proteins, heme oxygenase, histones, insulin, interferons, metallothioneins, nuclear hormone receptors, phenylethanolamine N-methyltransferase, phosphoglycerate kinase, prostate specific antigen, protamines, pyruvate kinases, renins, SCG10, skeletal actins or troponins, sodium channel type 11, synapsin, testis-specific histone Hlt, thyroid receptor-pl, transferrin, tyrosine hydroxylase, vascular cellular adhesion molecule-1, von Willebrand factor). In other embodiments, the regulated promoter comprises a promoter from a virus e.g., adenoviruses, adeno-associated virus, human cytomegalovirus, Epstein-Barr virus and other herpes simplex viruses, lentiviruses, Moloney leukemia or sarcomavirus, mouse mammary tumor virus, polyoma or SV40 virus, Rous sarcoma virus, or vaccinia virus. The regulated promoter may comprise any of the various promoters described above operably linked to an enhancer or other regulatory element, such as a response element, which regulates the expression of said promoter.

Another aspect of the invention provides a cell comprising a recombinant polymerase III promoter, wherein transcription from the recombinant polymerase III promoter increases when the cell is contacted with an ecdysone or an analog or mimic thereof. In a specific embodiments, the cell further comprises at least one of the following features:
1. A transgene operably linked to the polymerase III promoter, such as a transgene encoding an shRNA or an siRNA.
2. A recombinant transcription factor whose transcription is induced by ecdysone, which binds to a binding site operably linked to the polymerase III promoter and which promotes transcription from said promoter.
3. An ecdysone-inducible promoter which regulates the transcription of a recombinant transcription factor, wherein said transcription factor increases transcription from the polymerase III promoter.
4. One or more nucleic acids encoding one or more of (i) an ecdysone receptor, such as VgEcR, and (ii) an RXR nuclear receptor.

Another aspect of the invention provides a regulated polymerase III expression system, comprising a regulated promoter operably linked to a first element encoding a transcriptional repressor; and (b) a recombinant polymerase III promoter regulated by the transcription factor, wherein binding of the transcription factor to (i) the polymerase III promoter or to (ii) a binding site operably linked to the polymerase III promoter decreases transcription from the recombinant polymerase III promoter. In some embodiments, the regulated promoter is an inducible promoter, such that addition of an inducer, such as to a cell comprising the expression system, results in transcription of the transcriptional repressor. In preferred specific embodiments, the transcriptional repressor does not bind to the inducer, and its binding affinity for the polymerase III promoter or for a binding site operably linked to said promoter is substantially the same in the presence or absence of the inducer. In other embodiments, the transcriptional repressor does not comprise a tet DNA binding domain, while in other embodiments the transcriptional repressor does not bind to the tet operator. An expression system comprising a transcriptional repressor and comprising an inhibitory RNA under the control of the polymerase III promoter allows constitutive transcription of the RNA until an inducer is added. In such case, the gene expression of a gene can be suppressed until addition of the inducer.

In one embodiment of the regulated polymerase III expression systems described herein, the transcription factor comprises a DNA-binding domain and a transactivating domain. In some embodiments, the DNA-binding domain and the transactivation domain of the transcription factor are derived from two different proteins. Two domains from different proteins may be operably-joined into one polypeptide using well-known recombinant techniques. In a specific embodiment, the DNA-binding domain is a GAL4 DNA-binding domain. In another specific embodiment, the transactivation domain comprises an activation domain derived from an Octamer protein, such as an Oct-1 or Oct-2 transactivation domain. In some embodiments, the transactivation domain preferentially promotes transcription of polymerase III over that of polymerase I or polymerase II. Accordingly, in preferred embodiments, the transactivation domain is polymerase III specific.

In another specific embodiment, the transactivation domain comprises an Oct-2^{Q}domain. In another embodiment, the transcription factor comprises a mutant Oct-2^{Q}(Q→A). In another embodiment, the transactivation domain comprises an Oct-1 domain. Oct-1 and Oct-2, and mutants thereof, are described, for example, in Das et al. Nature 1995. 37: 657-660.

In one embodiment of the regulated polymerase III expression systems described herein, the transcription factor does not promote transcription from the polymerase III promoter unless it forms a multimer with a second protein. For example, in one embodiment, the transcription factor forms a heterodimer with a second transcription factor, and the heterodimer then promotes transcription from the polymerase III promoter. Examples of heterodimeric transcription factors are well known in the art, such transcription factors having zinc-finger, leucine zipper domains or basic helix-loop-helix domains. The second transcription factor may be constitutively expressed, or it may be expressed under the control of a second regulated promoter. The second regulated promoter may be active under the same conditions as the regulated promoter which transcribes the first the transcription factor. For example, both promoters may be induced in the presence of ecdysone hormone or an analog or mimic thereof, such that both transcription factors are expressed at the same time. Alternatively, the second transcription factor may be expressed under the control of a second regulated promoter. For example, the second regulated promoter may be a tissue specific promoter or a developmentally-regulated promoter, while the first regulated promoter is under the control of an inducer, such as ecdysone hormone or an agonist thereof. In embodiments where two transcriptions factors are present which regulate transcription from the polymerase III promoter, both transcription factors need not have transactivation domains.

In a specific embodiment, the second transcription factor in the absence of the first transcription factor inhibits transcription from the polymerase III promoter, such as when the second transcription factor forms a homodimer, but promotes transcription when complexed in a heterodimer with the first transcription factor. Such embodiment may provide yet a lower level of transcription in the absence of an inducer.

In a related embodiment, the first transcription factor and the second transcription factor both promote transcription from the polymerase III promoter, but they each bind weakly to DNA. When both factors are present, they exhibit cooperative binding and thus transcription form the polymerase III promoter is greatest in the presence of both factors. Such interactions have been described, for example, for NFAT and the AP-1 complex (Chen et al., 1998, Nature 392: 42).

In one embodiment of the regulated polymerase III expression systems described herein, the transcription factor binds to a site operably linked to the polymerase III promoter, while in other embodiments it binds to at least one. Similarly, one embodiment of the regulated polymerase III expression systems described herein comprises at least one binding site for the transcription factor to bind, said binding sites being operably linked to the recombinant polymerase III promoter. In preferred embodiments, binding of the transcription factor to the binding site increases transcription from the polymerase III promoter. In another embodiment, the regulated polymerase III expression system comprises four binding sites for the transcription factor, said binding sites being operably linked to the polymerase III promoter.

The number of binding sites operably linked to the polymerase III promoter and the spacing and/or distribution of binding sites relative to the polymerase III promoter may be adjusted by one skilled in the art for the desired level of basal expression and maximal expression. For example, and increased number of binding sites may result in both a greater basal and a greater maximal level of transcription from the polymerase III promoter. Conversely, a lower number of binding sites would be expected to result in a lower basal level and a lower induced level of transcription from the polymerase III promoter. By basal level it is meant the level of transcription in the absence of the transcription factor or in the presence of a minimal amount of transcription factor that may exist in the cell under conditions in which expression of the transcription factor is intended to be minimal. For example, in embodiments when the transcription factor is transcribed from a regulated promoter that is inducible, such as in the presence of the inducer Muristerone A, the basal level of transcription refers to the level of transcription in the absence of exogenously added inducer. By maximal transcription it is meant the maximal level of transcription from the polymerase III promoter achieved when the regulated promoter is active, such as in the presence of an inducer. As used herein, the signal-to-noise ratio of transcription from the polymerase III promoter refers to the ratio of maximal transcription to basal transcription.

In one embodiment, the regulated polymerase III expression systems described herein comprises a number of binding sites for the transcription factor that results in the lowest basal transcription, in the highest maximal transcription, or in the highest signal-to-noise ratio of transcription from the recombinant polymerase III promoter. In other embodiments, the number of binding sites is 1, 2, 3, 4, 5, 6, 7, 8, or 9.

In other embodiments, the signal-to-noise to ratio of transcription from the recombinant polymerase III promoter in the systems described herein is at least 2, 3, 4, 5, 6, 7, 8, 9, 10, 12, 14, 16, 18, 20, 25, 30, 40, 50, 60, 70, 80, 90, 100, 200, or 500.

In one embodiment of the regulated polymerase III expression systems described herein, the recombinant polymerase III promoter comprises a mammalian promoter. In specific embodiments, the polymerase III promoter comprises a U6 promoter, an H1 promoter, a 7SK promoter (Boyd et al., 2000. Gene 244: 33-44), or a tRNA promoter, such as a tRNA-met or a tRNA-val promoter. In one specific embodiment, a modified methionine tRNA promoter is used as described in Boden et al., 2003, Nucleic Acids Res. 31(17): 5033-8.

In one embodiment of the regulated polymerase III expression systems described herein, the recombinant polymerase III promoter comprises the enhancer from the cytomegalovirus immediate-early promoter. This enhancer has been shown to increase transcription from the U6 promoter (Xia et al. 2003 Nucleic Acids Res. 31(17): 100).

In another embodiment of the regulated polymerase III expression systems described herein, the recombinant polymerase III promoter comprises a TATA Box. In a specific embodiment, the TATA Box comprises the sequence TATAAA. In some embodiments, the binding site for the transcription factor does not comprise a TATA Box, a mutant TATA box which differs by one nucleotide substitution from the sequence TATAAA, or a mutant TATA Box comprising the sequence GTATAAA.

In another embodiment, the polymerase III promoter lacks endogenous enhancers. In another embodiment, the sequence(s) of endogenous enhancers are removed, such as by point mutations or deletions. In some embodiments, endogenous enhancers are replaced by binding sites for the transcription factor, such that the binding sites are operably linked to the promoter. In specific embodiments, the binding sites comprise GAL-4 DNA-binding sites.

One embodiment of the regulated polymerase III expression systems described herein comprises a second recombinant polymerase III promoter. The second RNA polymerase promoter may be a constitutive polymerase III promoter or it may be regulated by the transcription factor encoded by the first element. A regulated polymerase III promoter system comprising two or more recombinant polymerase III promoters, wherein each polymerase III promoter is operably linked to a transgene, allows for the coordinate expression of multiple transgenes.

In yet other embodiments, the recombinant polymerase III promoter comprises Pol III human or murine U6 and H1 systems, the cytomegalovirus (CMV) promoter/enhancer, the human β-actin promoter, the glucocorticoid-inducible promoter present in the mouse mammary tumor virus long terminal repeat (MMTV LTR), the long terminal repeat sequences of Moloney murine leukemia virus (MULV LTR), the SV40 early or late region promoter, the promoter contained in the 3′ long terminal repeat of Rous sarcoma virus (RSV), the herpes simplex virus (HSV) thymidine kinase promoter/enhancer, and the herpes simplex virus LAT promoter.

In some embodiments of the regulated polymerase III expression systems described herein, the regulated promoter is further operably linked to the a second element. The second element may encode a RNA of interest, such as an inhibitory RNA, or it may encode a polypeptide.

In some embodiments, the second element encodes a reporter protein. In certain applications, a reporter protein may be desirable to indicate the transcriptional state of the regulated promoter. In some embodiments, the reporter protein comprises a fluorescent or bioluminescent protein, such as a Green Flourescent Protein (GFP) or a mutant thereof. GFP mutants comprise those carrying one or more point mutations which shift the absorption or emission spectra of the GFP protein towards other wavelengths. GFP mutants include those described in Haseloff J. 1999. Methods Cell Biol.; 58: 139-51 and Prendergast F G 1999. Methods Cell Biol. 58: 1-18. In other embodiments, the reporter protein comprises drFP583 or DsRed (Matz et al., 1999. Nat Biotech 17: 969-973).

In some embodiments, the second element encodes a selectable marker. Selectable markers comprise proteins that confer drug-resistance, such as but not limited to those conferring resistance to hygromycin (Hyg), neomycin (Neo), puromycin (PAC), blasticidin S (BlaS) or Zeocin (Zeo). Selectable markers further comprise proteins which are expressed at the cell surface. Particularly useful proteins expressed at the cell surface comprise cell-surface receptors include those for which antibodies are commonly available, such as the CD4 and CD8 proteins, or other proteins which allow efficient sorting by immunological or affinity techniques, such as FACs sorting. In some embodiments, the second element encodes an enzyme. In some embodiments, the selectable marker is chloroamphenicol acetyl transferase, dihydrofolate reductase (DHFR), HSV-tk, lacZ or luciferase.

In another embodiment, the second element encodes a second transcription factor or a protein that can form a complex, such as a heterodimer, with the transcription factor. In some embodiments, the second transcription factor regulates transcription of the regulated promoter, such as by binding to the regulated promoter or to a binding site operably linked to said promoter, whereas in other embodiments the second transcription factor regulates transcription of the recombinant polymerase III promoter.

In some embodiments, the second element encodes a second transcription factor, a transcriptional activator or a transcriptional repressor. In specific embodiments, the transcription factors encoded by the first and second elements synergize to promote transcription from the polymerase III promoter, such as by exhibiting cooperative binding to DNA or by forming a hetero-multimer, such as a heterodimer. In another embodiment, the second transcription factor promotes transcription from the regulated promoter, such as by binding to the regulated promoter or to a site operably linked to the regulated promoter. Such embodiment can provide a positive feedback loop, such that when the regulated promoter is activated, the second transcription factor will promote further transcription from the regulated promoter. In such as system, the second transcription factor may act to lock the regulated promoter on, such that when the regulated promoter is turned on it will stay on, even in the absence of the original inducer in the case when the regulated promoter is an inducible promoter.

In another specific embodiment, the second element encodes a transcriptional repressor. In certain embodiments, the transcriptional repressor inhibits transcription from the regulated promoter, such as by binding to the regulated promoter and inhibiting recruitment of polymerase III to the promoter or by decreasing binding of a transcriptional activator to the polymerase III promoter. In such embodiments, the transcriptional repressor may act to provide a negative feedback loop. Such negative feedback loop may provide attenuated activation of the polymerase III promoter in response to activation of the regulated promoter.

In one embodiment, the regulated polymerase III expression systems further comprises the sequence of a transgene operably linked to the recombinant polymerase III promoter, such that transcription of the transgene regulated by the polymerase III promoter. In preferred embodiments, the transgene encodes a non-coding RNA. Non-coding RNAs may be inhibitory RNAs. In a specific embodiment, the non-coding RNA comprises an siRNA or a short hairpin RNA (shRNA). In some embodiments, the siRNA or shRNA effects the knockdown of a gene or of multiple genes. In some embodiments, the gene encodes a protein which is essential for cell survival. In a related embodiment, expression of the siRNA or shRNA a cell results in lethality of the cell, cell-cycle arrest or apoptosis. In embodiments in which a regulated polymerase expression system is found in multiple cells in an animal, the transgenes may be such that its expression in one, more than one, or in all of the cells all the animals result in lethality of the cell.

In some embodiments, the transgene comprises a ribozyme. In specific embodiments, the ribozyme comprises a Cech-type ribozyme or a hammerhead ribozyme. In other embodiment, the transgene encodes an RNA molecule that can be processed in vivo to generate shRNAs or siRNAs.

In one embodiment, the regulated polymerase III expression systems described herein further comprise a cloning site 3′ to the polymerase III promoter. In preferred embodiments, the position of the cloning site relative to the polymerase III promoter allows transcription of a DNA sequence inserted into the cloning site from the recombinant polymerase III promoter. In some embodiments, the cloning site comprises a restriction-enzyme recognition site. In another embodiment, the cloning site comprises a recombinase recognition site, such as a ccdB sequence.

In one embodiment, the regulated polymerase III expression systems described herein may be introduced into a cell without reduction in cell viability. In a further embodiment, induction of the regulated promoter by an inducer does not result in a reduction in cell viability.

Some embodiments of the regulated polymerase III expression systems described herein comprise at least one nucleic acid encoding a regulatory protein which promotes transcription from the regulated promoter. Similarly, other embodiments comprise at least one nucleic acid encoding two regulatory proteins which promote transcription from the regulated promoter. Still other embodiments comprise two nucleic acids each encoding a regulatory protein which promotes transcription from the regulated promoter. In some embodiments, the regulated protein acts as a transcriptional repressor under some conditions, such as in the absence of an inducer, and as a transcriptional activator under other conditions, such as in the presence of the inducer.

In some embodiments, the regulatory protein binds an inducer. In specific embodiments, binding of the regulatory protein to the inducer results in increased transcription from the regulated promoter. This may occur, for example, through, binding of the regulatory protein to the regulated promoter, or to at least one response element operably linked to the regulated promoter, upon binding of the inducer. Similarly, in one embodiment of the regulated polymerase III expression systems described herein, binding of an inducer to the regulatory protein promotes binding of the regulatory protein to the regulated promoter or to a response element operably-linked to said promoter. In yet other embodiments, binding of the regulatory protein to the regulated promoter or to a response element operably linked to the regulated promoter promotes transcription from the regulated promoter.

In some embodiments, the regulatory protein complexes with a second regulatory protein to form a heteromultimer. In specific embodiments, binding of an inducer by the regulatory protein, by a second regulatory protein, or by both, induces the formation of a heteromultimer, which then activates transcription from the regulated promoter, such as by binding to a response element operably linked to the regulated promoter or by binding to the regulated promoter. In other embodiments, the regulatory protein forms a homodimer, or a homomultimer, in the presence of an inducer. In specific embodiments, formation of a homodimer or a homomultimer promotes its binding to the regulated promoter, its binding to a response element operably liked to regulated promoter, increasing transcription from the regulated promoter, or a combination thereof.

In some embodiments, the regulatory protein comprises a DNA binding domain. In one embodiment, the DNA-binding domain binds to a response element operably linked to the regulated promoter, while in other embodiments the DNA-binding domain binds to the regulated promoter. In some embodiments, the DNA-binding domain of the regulatory protein comprises a tet repressor DNA-binding domain, an RxR DNA binding domain or a nuclear hormone receptor DNA-binding domain. In other embodiments, the DNA-binding domain of the regulatory protein forms a homodimer or a homomultimer in the presence of an inducer.

In preferred embodiments, the regulatory protein comprises an ecdysone receptor, a steroid hormone receptor, a nuclear hormone receptor, a transcriptional activator of a transcriptional repressor. Ecdysone receptors, ligands which bind to said receptors, and response elements to which the Ecdysone receptors bind have been described in the art, such as in U.S. Pat. Nos. 6,333,318, 6,245,531, 6,610,828, 6,258,603, in international PCT publication Nos. WO/9637609, WO/0170816, WO/02066612, WO/02066614, and in U.S. Patent Publication Nos. 2001/0044151, 2003/0110528, 2003/0088890. In specific embodiments, the regulatory protein comprises a VgEcR. In another specific embodiment, the regulatory protein comprises an retinoid X receptor (RXR) protein. In yet another specific embodiment, the regulated polymerase system described herein comprises one or more nucleic acids nucleic acid encoding an RXR nuclear receptor and an ecdysone receptor such as VgEcR. In a preferred embodiment, the RXR nuclear receptor and VgEcR heterodimerize in the presence of an inducer, and the resulting heterodimer binds to an ecdysone response element which is operably linked to the regulated promoter, such that binding results in increased transcription from the regulated promoter.

In one embodiment, the inducer comprises an ecdysone hormone, an or analog or mimic thereof, such as Muristerone A or Ponasterone A. In one embodiment, the ecdysone analog comprises ponasterone A, ponasterone B, ponasterone C, 26-iodoponasterone A, muristerone A, inokosterone or 26-mesylinokosterone. In another embodiment, the ecdysone mimic comprises 3,5-di-tert-butyl-4-hydroxy-N-isobutyl-benzamide, 8-O-acetylharpagide, a 1,2-diacyl hydrazine, an N′-substituted-N,N′-disubstituted hydrazine, a dibenzoylalkyl cyanohydrazine, an N-substituted-N-alkyl-N,N-diaroyl hydrazine, an N-substituted-N-acyl-N-alkyl, carbonyl hydrazine or an N-aroyl-N′-alkyl-N′-aroyl hydrazine. In yet another embodiment, the inducer comprises tetracycline, doxycycline, RU486, rapamycin, progesterone, or an analogs or mimetics thereof.

In some embodiments of the methods described herein, the regulatory protein is expressed in a tissue specific manner. For example, a regulatory protein comprising an ecdysone responsive transcription factor may be expressed in a specific tissue, such as in the liver of an animal such as a mouse. Administration of an ecdysone or an analog or mimic thereof to the animal results in transcription of the regulated promoter in the liver, and thus expression from the polymerase II promoter occurs primarily in the liver. Such embodiments result in both tissue specific and inducible expression from the polymerase III promoter. Likewise, the regulatory protein may be expressed under any other type of regulated promoter, such that expression from the polymerase III promoter is more specifically regulated.

The regulatory protein used in the regulated polymerase III expression systems described herein may comprise any transcription factor whose transcriptional activity or DNA binding activity is regulated by an inducer, including tet-repressor based transcriptional activators and repressors, progesterone receptors, or ecdysone receptors. Furthermore, one skilled in the art may, using standard recombinant techniques commonly known in the art, combine transactivation or repressor domains, DNA binding domains, and ligand or inducer bonding domains to generate transcription factors for a specific purpose.

IV. Nucleic Acids, Cells, Organisms and Kits

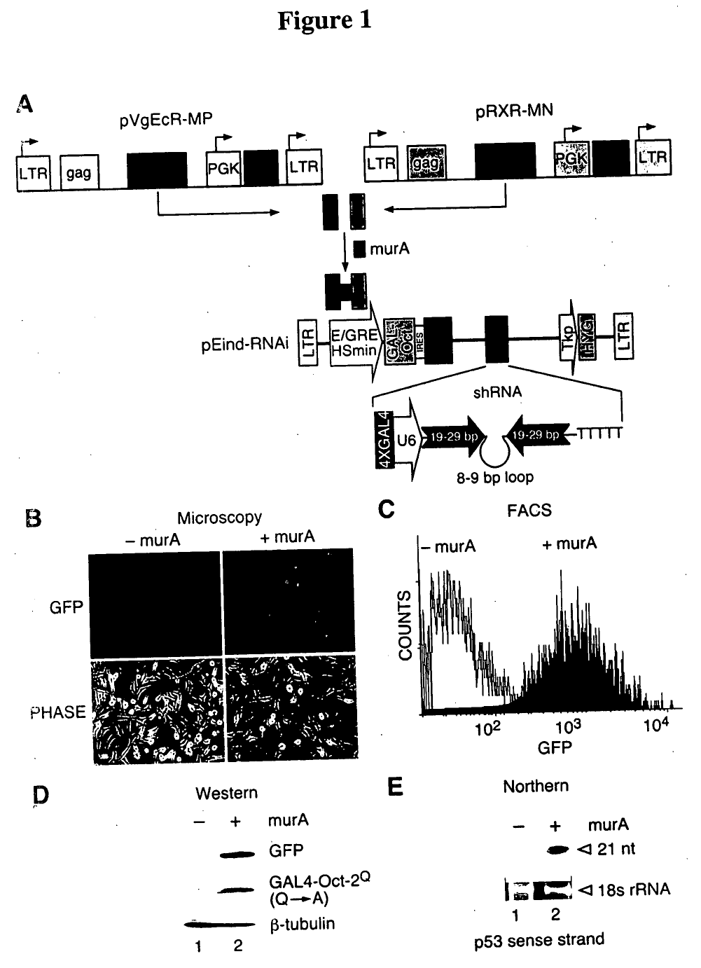

The invention further provides nucleic acids, cells and non-human transgenic organism comprising any of the components of the regulated polymerase III expression systems described herein. A nucleic acids provided by the invention may comprise multiple components of the regulated polymerase III expression systems described herein or they may contain only one component. For example, as illustrated in FIG. 1a, the invention provides in one embodiment a nucleic aid comprising the VgEcR and RXR components, while another nucleic acid comprises the regulated promoter and the first and second elements. Conversely, all components of the polymerase III expression system can be inserted into a single nucleic acid.

One aspect of the invention provides a nucleic acid comprising at least one variant of the inducible polymerase III expression systems described herein. In some embodiments, the nucleic acids which comprise the components of the regulated polymerase III expression system are provided as plasmids or viral vectors. In some embodiments, the invention provides nucleic acid sequences which comprise SEQ ID NO:1 or SEQ ID NO: 2.

Another aspect of the invention provides a cell comprising any of the regulated polymerase III expression systems described herein or components thereof, such as a cell comprising nucleic acids encoding the components of the polymerase III expression systems. The cell having regulated polymerase III expression system may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

The invention further provides viruses comprising any of the regulated polymerase III expression systems described herein or components thereof. Of particular interest are viruses capable of transforming mammalian cells.

Essentially any method for introducing any of the nucleic acids described herein into cells may be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle may be used to accomplish both efficient introduction of an expression construct into the cell and transcription of the components of the polymerase III expression system. Other methods known in the art for introducing nucleic acids to cells may be used, such as lipid-mediated carrier transport, chemical mediated transport, such as calcium phosphate, and the like. Thus the shRNA-encoding nucleic acid construct may be introduced along with components that perform one or more of the following activities: enhance RNA uptake by the cell, promote annealing of the duplex strands, stabilize the annealed strands, or otherwise increase inhibition of the target gene.

Another aspect of the invention provides nonhuman transgenic organism comprising nucleic acids comprising components of any of the regulated polymerase III expression systems described herein. In specific embodiments, the transgenic organism comprises a nucleic acid comprising the nucleotide sequence according to SEQ ID NO:1 or SEQ ID NO:2. The nucleic acids may be stably or transiently introduced into specific organisms using standard techniques known in the art for the generation of transgenic animals, including but not limited to introducing the nucleic acids into stem cells from which the organisms are derived.

Regulated polIII promoter systems may also be introduced into humans for therapeutic effect. For example, a regulated polIII-shRNA construct may be introduced in order to decrease expression of a disease related gene. Expression of the shRNA construct may be regulated by an exogenous factor, such as ecdysone or a tetracycline. Expression of the shRNA construct may also be regulated by cell or disease-specific promoters. For example, a shRNA construct may be designed to target viral genes and regulated so as to be expressed only in infected cells.

In general, regulated constructs may be introduced directly into a human or nonhuman organism, or may be introduced first into a cell, such as a stem cell, which is then introduced into the organism. For direct introduction, a variety of vector and transfection systems are known in the art. See, for example, Dubensky et al. (1984) Proc. Natl. Acad. Sci. USA 81, 7529-7533; Kaneda et al., (1989) Science 243, 375-378; Hiebert et al. (1989) Proc. Natl. Acad. Sci. USA 86, 3594-3598; Hatzoglu et al. (1990) J. Biol. Chem. 265, 17285-17293 and Ferry, et al. (1991) Proc. Natl. Acad. Sci. USA 88, 8377-8381. The vector may be administered by injection, e.g. intravascularly or intramuscularly, inhalation, or other parenteral mode. Non-viral delivery methods such as administration of the DNA via complexes with liposomes or by injection, catheter or biolistics may also be used.

In general, the manner of introducing the nucleic acid will depend on the nature of the tissue, the efficiency of cellular modification required, the number of opportunities to modify the particular cells, the accessibility of the tissue to the nucleic acid composition to be introduced, and the like. The DNA introduction need not result in integration. In fact, non-integration often results in transient expression of the introduced DNA, and transient expression is often sufficient or even preferred.

Any means for the introduction of polynucleotides into mammals, human or non-human, may be adapted to the practice of this invention for the delivery of the various constructs of the invention into the intended recipient. In one embodiment of the invention, the nucleic acid constructs are delivered to cells by transfection, i.e., by delivery of “naked” nucleic acid or in a complex with a colloidal dispersion system. A colloidal system includes macromolecule complexes, nanocapsules, microspheres, beads, and lipid-based systems including oil-in-water emulsions, micelles, mixed micelles, and liposomes. An exemplary colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10: 24-29, 1994; Tsan et al, Am J Physiol 268; Alton et al., Nat Genet. 5: 135-142, 1993 and U.S. Pat. No. 5,679,647 by Carson et al.

Optionally, liposomes or other colloidal dispersion systems are targeted. Targeting can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. A certain level of targeting may be achieved through the mode of administration selected.

In certain variants of the invention, the nucleic acid constructs are delivered to cells, and particularly cells in an organism or a cultured tissue, using viral vectors. The transgene may be incorporated into any of a variety of viral vectors useful in gene therapy, such as recombinant retroviruses, adenovirus, adeno-associated virus (AAV), herpes simplex derived vectors, hybrid adeno-associated/herpes simplex viral vectors, influenza viral vectors, especially those based on the influenza A virus, and alphaviruses, for example the Sinbis and semliki forest viruses, or recombinant bacterial or eukaryotic plasmids. The following additional guidance on the choice and use of viral vectors may be helpful to the practitioner. As described in greater detail below, such embodiments of the subject expression constructs are specifically contemplated for use in various in vivo and ex vivo gene therapy protocols.

A variety of herpes virus-based vectors have been developed for introduction of genes into mammals. For example, herpes simplex virus type 1 (HSV-1) is a human neurotropic virus of particular interest for the transfer of genes to the nervous system. After infection of target cells, herpes viruses often follow either a lytic life cycle or a latent life cycle, persisting as an intranuclear episome. In most cases, latently infected cells are not rejected by the immune system. For example, neurons latently infected with HSV-1 function normally and are not rejected. Some herpes viruses possess cell-type specific promoters that are expressed even when the virus is in a latent form.

A typical herpes virus genome is a linear double stranded DNA molecule ranging from 100 to 250 kb. HSV-1 has a 152 kb genome. The genome may include long and short regions (termed UL and US, respectively) which are linked in either orientation by internal repeat sequences (IRL and IRS). At the non-linker end of the unique regions are terminal repeats (TRL and TRS). In HSV-1, roughly half of the 80-90 genes are non-essential, and deletion of non-essential genes creates space for roughly 40-50 kb of foreign DNA (Glorioso et al, 1995). Two latency active promoters which drive expression of latency activated transcripts have been identified and may prove useful for vector transgene expression (Marconi et al, 1996).

HSV-1 vectors are available in amplicons and recombinant HSV-1 virus forms. Amplicons are bacterially produced plasmids containing OriC, an Escherichia coli origin of replication, OriS (the HSV-1 origin of replication), HSV-1 packaging sequence, the transgene under control of an immediate-early promoter & a selectable marker (Federoff et al, 1992). The amplicon is transfected into a cell line containing a helper virus (a temperature sensitive mutant) which provides all the missing structural and regulatory genes in trans. More recent amplicons include an Epstein-Barr virus derived sequence for plasmid episomal maintenance (Wang & Vos, 1996). Recombinant viruses are made replication deficient by deletion of one the immediate-early genes e.g. ICP4, which is provided in trans. Deletion of a number of immediate-early genes substantially reduces cytotoxicity and allows expression from promoters that would be silenced in the wild type latent virus. These promoters may be of use in directing long term gene expression. Replication-conditional mutants replicate in permissive cell lines. Permissive cell lines supply a cellular enzyme to complement for a viral deficiency. Mutants include thymidine kinase (During et al, 1994), ribonuclease reductase (Kramm et al, 1997), UTPase, or the neurovirulence factor g34.5 (Kesari et al, 1995). These mutants are particularly useful for the treatment of cancers, killing the neoplastic cells which proliferate faster than other cell types (Andreansky et al, 1996, 1997). A replication-restricted HSV-1 vector has been used to treat human malignant mesothelioma (Kucharizuk et al, 1997). In addition to neurons, wild type HSV-1 can infect other non-neuronal cell types, such as skin (Al-Saadi et al, 1983), and HSV-derived vectors may be useful for delivering transgenes to a wide array of cell types. Other examples of herpes virus vectors are known in the art (U.S. Pat. No. 5,631,236 and WO 00/08191).

Adenoviral systems may also be used. Knowledge of the genetic organization of adenovirus, a 36 kB, linear and double-stranded DNA virus, allows substitution of a large piece of adenoviral DNA with foreign sequences up to 8 kB. In contrast to retrovirus, the infection of adenoviral DNA into host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. In addition, adenoviral vector-mediated transfection of cells is often a transient event. A combination of immune response and promoter silencing appears to limit the time over which a transgene introduced on an adenovirus vector is expressed.

Adenovirus is suitable for use as a gene transfer vector in part because of its mid-sized genome, ease of manipulation, high titer, wide target-cell range, and high infectivity. The virus particle is relatively stable and amenable to purification and concentration, and as above, can be modified so as to affect the spectrum of infectivity. Additionally, adenovirus is easy to grow and manipulate and exhibits broad host range in vitro and in vivo. This group of viruses can be obtained in high titers and they are highly infective. Moreover, the carrying capacity of the adenoviral genome for foreign DNA is large (up to 8 kilobases) relative to other gene delivery vectors (Berkner et al., supra; Haj-Ahmand and Graham (1986) J. Virol. 57: 267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16: 683; Berkner et al., supra; and Graham et al., in Methods in Molecular Biology, E. J. Murray, Ed. (Humana, Clifton, N.J., 1991) vol. 7. pp. 109-127). Expression of the inserted polynucleotide of the invention can be under control of, for example, the E1A promoter, the major late promoter (MLP) and associated leader sequences, the viral E3 promoter, or exogenously added promoter sequences.

The genome of an adenovirus can be manipulated such that it encodes a gene product of interest, but is inactivated in terms of its ability to replicate in a normal lytic viral life cycle (see, for example, Berkner et al., (1988) BioTechniques 6: 616; Rosenfeld et al., (1991) Science 252: 431-434; and Rosenfeld et al., (1992) Cell 68: 143-155). Suitable adenoviral vectors derived from the adenovirus strain Ad type 5 d1324 or other strains of adenovirus (e.g., Ad2, Ad3, Ad7 etc.) are well known to those skilled in the art.

Adenoviruses can be cell type specific, i.e., infect only restricted types of cells and/or express a transgene only in restricted types of cells. For example, the viruses may be engineered to comprise a gene under the transcriptional control of a transcription initiation region specifically regulated by target host cells, as described e.g., in U.S. Pat. No. 5,698,443, by Henderson and Schuur, issued Dec. 16, 1997. Thus, replication competent adenoviruses can be restricted to certain cells by, e.g., inserting a cell specific response element to regulate a synthesis of a protein necessary for replication, e.g., E1A or E1B.

DNA sequences of a number of adenovirus types are available from Genbank. For example, human adenovirus type 5 has GenBank Accession No. M73260. The adenovirus DNA sequences may be obtained from any of the 42 human adenovirus types currently identified. Various adenovirus strains are available from the American Type Culture Collection, Rockville, Md., or by request from a number of commercial and academic sources. A transgene as described herein may be incorporated into any adenoviral vector and delivery protocol, by restriction digest, linker ligation or filling in of ends, and ligation.

Adenovirus producer cell lines can include one or more of the adenoviral genes E1, E2a, and E4 DNA sequence, for packaging adenovirus vectors in which one or more of these genes have been mutated or deleted are described, e.g., in PCT/US95/15947 (WO 96/18418) by Kadan et al.; PCT/US95/07341 (WO 95/346671) by Kovesdi et al.; PCT/FR94/00624 (WO94/28152) by Imler et al.; PCT/FR94/00851 (WO 95/02697) by Perrocaudet et al., PCT/US95/14793 (WO96/14061) by Wang et al.

Yet another viral vector system useful for delivery of the subject polynucleotides is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158: 97-129).

AAV has not been associated with the cause of any disease. AAV is not a transforming or oncogenic virus. AAV integration into chromosomes of human cell lines does not cause any significant alteration in the growth properties or morphological characteristics of the cells. These properties of AAV also recommend it as a potentially useful human gene therapy vector.

AAV is also one of the few viruses that may integrate its DNA into non-dividing cells, e.g., pulmonary epithelial cells, and exhibits, a high frequency of stable integration (see for example Flotte et al., (1992) Am. J. Respir. Cell. Mol. Biol. 7: 349-356; Samulski et al., (1989) J. Virol. 63: 3822-3828; and McLaughlin et al., (1989) J. Virol. 62: 1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5: 3251-3260 can be used to introduce DNA into cells. A variety of nucleic acids have been introduced into different cell types using AAV vectors (see for example Hermonat et al., (1984) PNAS USA 81: 6466-6470; Tratschin et al., (1985) Mol. Cell. Biol. 4: 2072-2081; Wondisford et al., (1988) Mol. Endocrinol. 2: 32-39; Tratschin et al., (1984) J. Virol. 51: 611-619; and Flotte et al., (1993) J. Biol. Chem. 268: 3781-3790).

The AAV-based expression vector to be used typically includes the 145 nucleotide AAV inverted terminal repeats (ITRs) flanking a restriction site that can be used for subcloning of the transgene, either directly using the restriction site available, or by excision of the transgene with restriction enzymes followed by blunting of the ends, ligation of appropriate DNA linkers, restriction digestion, and ligation into the site between the ITRs. The capacity of AAV vectors is usually about 4.4 kb (Kotin, R. M., Human Gene Therapy 5: 793-801, 1994 and Flotte, et al. J. Biol. Chem. 268: 3781-3790, 1993).

AAV stocks can be produced as described in Hermonat and Muzyczka (1984) PNAS 81: 6466, modified by using the pAAV/Ad described by Samulski et al. (1989) J. Virol. 63: 3822. Concentration and purification of the virus can be achieved by reported methods such as banding in cesium chloride gradients, as was used for the initial report of AAV vector expression in vivo (Flotte, et al. J. Biol. Chem. 268: 3781-3790, 1993) or chromatographic purification, as described in O'Riordan et al., WO97/08298. Methods for in vitro packaging AAV vectors are also available and have the advantage that there is no size limitation of the DNA packaged into the particles (see, U.S. Pat. No. 5,688,676, by Zhou et al., issued Nov. 18, 1997). This procedure involves the preparation of cell free packaging extracts.

Hybrid Adenovirus-AAV vectors have been generated and are typically represented by an adenovirus capsid containing a nucleic acid comprising a portion of an adenovirus, and 5′ and 3′ inverted terminal repeat sequences from an AAV which flank a selected transgene under the control of a promoter. See e.g. Wilson et al, International Patent Application Publication No. WO 96/13598. This hybrid vector is characterized by high titer transgene delivery to a host cell and the ability to stably integrate the transgene into the host cell chromosome in the presence of the rep gene. This virus is capable of infecting virtually all cell types (conferred by its adenovirus sequences) and stable long term transgene integration into the host cell genome (conferred by its AAV sequences).

The adenovirus nucleic acid sequences employed in this vector can range from a minimum sequence amount, which requires the use of a helper virus to produce the hybrid virus particle, to only selected deletions of adenovirus genes, which deleted gene products can be supplied in the hybrid viral process by a packaging cell. For example, a hybrid virus can comprise the 5′ and 3′ inverted terminal repeat (ITR) sequences of an adenovirus (which function as origins of replication). The left terminal sequence (5′) sequence of the Ad5 genome that can be used spans bp 1 to about 360 of the conventional adenovirus genome (also referred to as map units 0-1) and includes the 5′ ITR and the packaging/enhancer domain. The 3′ adenovirus sequences of the hybrid virus include the right terminal 3′ ITR sequence which is about 580 nucleotides (about bp 35,353-end of the adenovirus, referred to as about map units 98.4-100).

For additional detailed guidance on adenovirus and hybrid adenovirus-AAV technology which may be useful in the practice of the subject invention, including methods and materials for the incorporation of a transgene, the propagation and purification of recombinant virus containing the transgene, and its use in transfecting cells and mammals, see also Wilson et al, WO 94/28938, WO 96/13597 and WO 96/26285, and references cited therein.

Retroviral vectors may be employed in various embodiments of the invention. In most retroviral vectors, a nucleic acid of interest is inserted into the viral genome in the place of certain viral sequences to produce a virus that is replication-defective. In order to produce virions, a packaging cell line containing the gag, pol, and env genes but without the LTR and psi components may be constructed. When a recombinant plasmid containing a human cDNA, together with the retroviral LTR and psi sequences is introduced into this cell line (e.g., by calcium phosphate precipitation), the psi sequence allows the RNA transcript of the recombinant plasmid to be packaged into viral particles, which are then secreted into the culture media. The media containing the recombinant retroviruses is then collected, optionally concentrated, and used for gene transfer. Many retroviral vectors are able to infect a broad variety of cell types. Integration and stable expression require the division of host cells. This aspect is particularly relevant for the treatment of PVR, since these vectors allow selective targeting of cells which proliferate, i.e., selective targeting of the cells in the epiretinal membrane, since these are the only ones proliferating in eyes of PVR subjects.

A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76: 271). Thus, recombinant retrovirus can be constructed in which part of the retroviral coding sequence (gag, pol, env) has been replaced by nucleic acid encoding a protein of the present invention, e.g., a transcriptional activator, rendering the retrovirus replication defective. The replication defective retrovinis is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. A preferred retroviral vector is a pSR MSVtkNeo (Muller et al. (1991) Mol. Cell Biol. 11: 1785 and pSR MSV(XbaI) (Sawyers et al. (1995) J. Exp. Med. 181: 307) and derivatives thereof. For example, the unique BamHI sites in both of these vectors can be removed by digesting the vectors with BamHI, filling in with Klenow and religating to produce pSMTN2 and pSMTX2, respectively, as described in PCT/US96/09948 by Clackson et al. Examples of suitable packaging virus lines for preparing both ecotropic and amphotropic retroviral systems include Crip, Cre, 2 and Am.

Retroviruses, including lentiviruses, have been used to introduce a variety of genes into many different cell types, including neural cells, epithelial cells, retinal cells, endothelial cells, lymphocytes, myoblasts, hepatocytes, bone marrow cells, in vitro and/or in vivo (see for example, review by Federico (1999) Curr. Opin. Biotechnol. 10: 448; Eglitis et al., (1985) Science 230: 1395-1398; Danos and Mulligan, (1988) PNAS USA 85: 6460-6464; Wilson et al., (1988) PNAS USA 85: 3014-3018; Armentano et al., (1990) PNAS USA 87: 6141-6145; Huber et al., (1991) PNAS USA 88: 8039-8043; Ferry et al., (1991) PNAS USA 88: 8377-8381; Chowdhury et al., (1991) Science 254: 1802-1805; van Beusechem et al., (1992) PNAS USA 89: 7640-7644; Kay et al., (1992) Human Gene Therapy 3: 641-647; Dai et al., (1992) PNAS USA 89: 10892-10895; Hwu et al., (1993) J. Immunol. 150: 4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86: 9079-9083; Julan et al., (1992) J. Gen Virol 73: 3251-3255; and Goud et al., (1983) Virology 163: 251-254); or coupling cell surface ligands to the viral env proteins (Neda et al., (1991) J. Biol. Chem. 266: 14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Examples of other viral vector systems that can be used to deliver a polynucleotide of the invention have been derived from vaccinia virus, alphavirus, poxvirus, arena virus, polio virus, and the like. Such vectors offer several attractive features for various mammalian cells. (Ridgeway (1988) In: Rodriguez R L, Denhardt D T, ed. Vectors: A survey of molecular cloning vectors and their uses. Stoneham: Butterworth; Baichwal and Sugden (1986) In: Kucherlapati R, ed. Gene transfer. New York: Plenum Press; Coupar et al. (1988) Gene, 68: 1-10; Walther and Stein (2000) Drugs 60: 249-71; Timiryasova et al. (2001) J Gene Med 3: 468-77; Schlesinger (2001) Expert Opin Biol Ther 1: 177-91; Khromykh (2000) Curr Opin Mol Ther 2: 555-69; Friedmann (1989) Science, 244: 1275-1281; Ridgeway, 1988, supra; Baichwal and Sugden, 1986, supra; Coupar et al., 1988; Horwich et al. (1990) J. Virol., 64: 642-650).

Where a cell is to be introduced to a subject, an appropriate method may be selected, depending on the cell type. In certain embodiments, the invention provides a composition formulated for administration to a patient, such as a human or veterinary patient. A composition so formulated may comprise a stem cell comprising a nucleic acid construct encoding a regulated polIII system, such as a regulated shRNA construct for gene silencing. A composition may also comprise a pharmaceutically acceptable excipient. Essentially any suitable cell may be used, included cells selected from among those disclosed herein. Transfected cells may also be used in the manufacture of a medicament for the treatment of subjects. Examples of pharmaceutically acceptable excipients include matrices, scaffolds or other substrates to which cells may attach (optionally formed as solid or hollow beads, tubes, or membranes), as well as reagents that are useful in facilitating administration (e.g. buffers and salts), preserving the cells (e.g. chelators such as sorbates, EDTA, EGTA, or quaternary amines or other antibiotics), or promoting engraftment.

Cells may be encapsulated in a membrane or in a microcapsule. Cells may be placed in microcapsules composed of alginate or polyacrylates. (Lim et al. (1980) Science 210: 908; O'Shea et al. (1984) Biochim. Biochys. Acta. 840: 133; Sugamori et al. (1989) Trans. Am. Soc. Artif. Intern. Organs 35: 791; Levesque et al. (1992) Endocrinology 130: 644; and Lim et al. (1992) Transplantation 53: 1180). Additional methods for encapsulating cells are known in the art. (Aebischer et al. U.S. Pat. No. 4,892,538; Aebischer et al. U.S. Pat. No. 5,106,627; Hoffman et al. (1990) Expt. Neurobiol. 110: 39-44; Jaeger et al. (1990) Prog. Brain Res. 82: 41-46; and Aebischer et al. (1991) J. Biomech. Eng. 113: 178-183, U.S. Pat. No. 4,391,909; U.S. Pat. No. 4,353,888; Sugamori et al. (1989) Trans. Am. Artif. Intern. Organs 35: 791-799; Sefton et al. (1987) Biotehnol. Bioeng. 29: 1135-1143; and Aebischer et al. (1991) Biomaterials 12: 50-55).

V. Methods

In certain aspects, the invention provides methods relating to the use of RNA interference to inducibly and reversibly decrease the expression of one or more target genes in cells. Recent work has shown that the RNA interference effects of exogenously provided dsRNAs can be recapitulated in mammalian cells by the expression of single RNA molecules which fold into stable “hairpin” structures (Paddison et al. Genes Dev 16(8): 948-58 (2002)). Transient transfection of plasmids encoding short “hairpin” RNAs (shRNAs) can achieve a near complete reduction in the levels of a specific protein in a cell. Applicants have now demonstrated that shRNAs can be inducibly and reversibly expressed in mammalian cells. A variety of experiments substantiating the discovery are presented in detail in the Experimental sections below.

Accordingly, in certain aspects, the invention provides methods of reducing gene expression. The regulated polymerase III expression systems described herein which comprise a transgene encoding an inhibitory RNA under the control of the polymerase III promoter may be introduced into a cell in vitro or in vivo, or into an organism, to reduce expression of a gene to which the inhibitory RNA is directed.

One aspect of the invention provides method of reducing expression of a gene in a cell, the method comprising providing a cell comprising any of regulated polymerase III expression systems described herein, wherein the recombinant polymerase III promoter is operably linked to a coding sequence for an RNA molecule and wherein expression of the RNA molecule reduces expression of the gene; and (b) contacting the cell with an inducer, wherein the inducer promotes transcription of the RNA molecule from the recombinant polymerase III promoter, thereby reducing expression of the gene in the cell.

Another aspect of the invention provides a method of reducing gene expression of a gene in a cell, the method comprising (a) providing a cell comprising (i) a regulated promoter operably linked to a first element encoding a transcription factor; and (ii) a recombinant polymerase III promoter regulated by the transcription factor and operably linked to a coding sequence for an RNA molecule, wherein expression of the RNA molecule reduces expression of the gene; and (b) contacting the cell with an inducer, wherein the inducer promotes transcription of the RNA molecule from the recombinant polymerase III promoter, thereby reducing expression of the gene in the cell.

In a preferred embodiment of the methods described herein, the RNA molecule comprises a short hairpin RNA (shRNA) molecule or an siRNA molecule. A double-stranded structure of an shRNA is formed by a single self-complementary RNA strand. RNA duplex formation may be initiated either inside or outside the cell. Inhibition is sequence-specific in that nucleotide sequences corresponding to the duplex region of the RNA are targeted for genetic inhibition. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Because 100% sequence identity between the RNA and the target gene is not required to practice the present invention, the invention has the advantage of being able to tolerate sequence variations that might be expected due to genetic mutation, strain polymorphism, or evolutionary divergence. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

In a preferred embodiment, a shRNA construct is designed with about 29 bp helices. An expression cassette comprising the polymerase III promoter and the transgene may be delivered to the cell via a Murine Stem Cell Virus (MSCV)-based retrovirus, with the expression cassette inserted downstream of the packaging signal. Further information on the optimization of shRNA constructs may be found, for example, in the following references: Paddison, et al. Proc Natl Acad Sci USA, 2002. 99(3): p. 1443-8; 13. Brummelkamp, et al. Science, 2002. 21: p. 21; Kawasaki, et al. Nucleic Acids Res, 2003. 31(2): p. 700-7; Lee et al. Nat Biotechnol, 2002. 20(5): p. 500-5; Miyagishi, et al. Nat Biotechnol, 2002. 20(5): p. 497-500; Paul., et al., Nat Biotechnol, 2002. 20(5): p. 505-8.

An shRNA will generally be designed to have partial or complete complementarity with one or more target genes (i.e., complementarity with one or more transcripts of one or more target genes). The target gene may be a gene derived from the cell, an endogenous gene, a transgene, or a gene of a pathogen which is present in the cell after infection thereof. Depending on the particular target gene, the nature of the shRNA and the level of expression of shRNA (e.g. depending on copy number, promoter strength) the procedure may provide partial or complete loss of function for the target gene. Quantitation of gene expression in a cell may show similar amounts of inhibition at the level of accumulation of target mRNA or translation of target protein.

“Inhibition of gene expression” refers to the absence or observable decrease in the level of protein and/or mRNA product from a target gene. “Specificity” refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

As disclosed herein, the present invention is not limited to any type of target gene or nucleotide sequence. In some preferred embodiments, the target gene is an essential gene or a gene which is essential for cell viability. The following classes of possible target genes are listed for illustrative purposes: developmental genes (e.g., adhesion molecules, cyclin kinase inhibitors, Writ family members, Pax family members, Winged helix family members, Hox family members, cytokines, lymphokines and their receptors, growth/differentiation factors and their receptors, neurotransmitters and their receptors); oncogenes (e.g., ABLI, BCLI, BCL2, BCL6, CBFA2, CBL, CSFIR, ERBA, ERBB, EBRB2, ETSI, ETS1, ETV6, FGR, FOS, FYN, HCR, HRAS, JUN, KRAS, LCK, LYN, MDM2, MLL, MYB, MYC, MYCLI, MYCN, NRAS, PIM 1, PML, RET, SRC, TALI, TCL3, and YES); tumor suppressor genes (e.g., APC, BRCA1, BRCA2, MADH4, MCC, NF 1, NF2, RB 1, P53, BIM, PUMA and WTI); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases).

An additional aspect of the invention provides method of determining the effects of reducing expression of a gene in a cell, the method comprising (a) providing a cell comprising (i) a regulated promoter operably linked to a first element encoding a transcription factor; and (ii) a recombinant polymerase III promoter regulated by the transcription factor and operably linked to a coding sequence for an RNA molecule, wherein expression of the RNA molecule reduces expression of the gene; (b) subjecting the cell to a condition which promotes transcription of the RNA molecule from the recombinant polymerase III promoter; and (c) determining the phenotype of the cell, thereby determining the effects of reducing expression of the gene. The condition to which the cell is subjected to promote transcription of the RNA molecule will depend on the type of regulated promoter. For example, if the regulated promoter is an inducible promoter, such as an ecdysone-inducible promoter, the cell can be contacted with an inducer, such as with an ecdysone. If the regulated promoter is induced by DNA damage, the cell may be treated with radiation or radiomimetic agents. If the regulated promoter is a chemokine-regulated promoter, the cell may be contacted with the appropriate chemokine. One skill in the art may determine the appropriate conditions to induced expression of the RNA molecule based on the factors that promote expression from the regulated promoter.

Yet another aspect of the invention provides a method of determining the effects of silencing expression of a gene in an organism, the method comprising (a) providing an organism wherein at least a cell in the organism comprises (i) a regulated promoter operably linked to a first element encoding a transcription factor; and (ii) a recombinant polymerase III promoter regulated by the transcription factor and operably linked to a coding sequence for an RNA molecule, wherein expression of the RNA molecule reduces expression of the gene; (b) subjecting the organism to conditions which promote transcription of the RNA molecule from the recombinant polymerase III promoter in at least one cell; and (c) determining the phenotype of at least one cell in the organism; thereby determining the effects of silencing expression of a gene in an organism.

In some embodiments of the methods described herein, the regulated promoter is an inducible promoter. In specific embodiments, transcription from the promoter is increased in the presence of an ecdysone, an ecdysone analog or an ecdysone mimic. In some embodiments, the transcription factor does not comprise a tet DNA binding domain or does not bind to an inducer, such as a tetracycline or doxycycline inducer. In preferred embodiments of the methods, the expression of the transcription factor is dependent on the presence of an inducer, preferably an ecdysone, an ecdysone analog or an ecdysone mimic.

In other embodiments of the methods described herein, the transcription factor regulates transcription from the recombinant RNA polymerase III promoter by binding to (i) a binding site operably linked to said promoter; or (ii) to said promoter. In specific embodiments, the binding affinity of the transcription factor for (i) the polymerase III promoter or for (ii) the binding site operably linked to said promoter is substantially the same in the presence or absence of the inducer. In other specific embodiments, binding of the transcription factor to the recombinant RNA polymerase promoter by or to a binding site operably linked to said promoter increases transcription from said promoter.

The invention also provides variations of the methods described herein, wherein gene expression of more than one gene is achieved. This may be achieved for example, by expressing multiple shRNAs, or by designing an shRNA to inhibit the gene expression of two or more genes which share substantial nucleotide sequence identity in a short stretch, preferably at least 90% identity over a length of 20, 22, 25, 27, or 30 nucleotides.

In some embodiments of the methods described herein, the cell is in an organism. In another embodiment, the cell is a stem cell. The organism may be any type of organism, including an animal, plant, fungus, mammal, or mouse.

As will be apparent to one of ordinary skill in the art upon review of this disclosure, methods and compositions described herein may be employed in essentially any situation in which it is desirable to regulate expression of an RNA silencing construct or other construct to be expressed from a polIII promoter. The following are illustrative examples of methods in which such technology may be employed.

In certain aspects, the invention provides methods of treating a disorder in a subject by introducing cells comprising a regulated shRNA expression construct. In accordance with methods disclosed herein, the shRNA may be expressed in vivo in a variety of cell types. In certain embodiments the cells are administered in order to treat a condition. There are a variety of mechanisms by which shRNA expressing cells may be useful for treating a condition. For example, a condition may be caused in part by a population of cells expressing an undesirable gene. These cells may be ablated and replaced with administered cells comprising shRNA, when expressed, decreases expression of the undesirable gene; alternatively, the diseased cells may be competed away by the administered cells, without need for ablation. As another example, a condition may be caused by a deficiency in a secreted factor. Amelioration of such a disorder may be achieved by administering cells expressing a shRNA that indirectly stimulates production of the secreted factor, e.g., by inhibiting expression of an inhibitor. A regulated shRNA construct may be designed for expression in only certain cell types or in response to specific agents supplied exogenously to the patient. Therefore, the use of regulated shRNA expression vectors can improve the temporal and/or spatial targeting of RNAi-based therapeutics.

A shRNA may be targeted to essentially any gene, the decreased expression of which may be helpful in treating a condition. The target gene participate in a disease process in the subject. The target gene may encode a host protein that is co-opted by a virus during viral infection, such as a cell surface receptor to which a virus binds while infecting a cell. HIV binds to several cell surface receptors, including CD4 and CXCR5. The introduction of HSCs or other T cell precursors carrying an shRNA directed to an HIV receptor or coreceptor is expected to create a pool of resistant T cells, thereby ameliorating the severity of the HIV infection. Similar principles apply to other viral infections.

Immune rejection is mediated by recognition of foreign Major Histocompatibility Complexes. Where heterologous cells are to be administered to a subject, the cells may be transfected with shRNAs that target any MHC components that are likely to be recognized by the host immune system.

In many embodiments, the shRNA transfected cells will achieve beneficial results by partially or wholly replacing a population of diseased cells in the subject. The transfected cells may autologous cells derived from cells of the subject, but carrying a shRNA that confers beneficial effects.

Certain regulated polIII promoters disclosed herein may be used in methods of identifying gene function in an organism, especially higher eukaryotes, comprising the use of double-stranded RNA to inhibit the activity of a target gene of previously unknown function. Instead of the time consuming and laborious isolation of mutants by traditional genetic screening, functional genomics would envision determining the function of uncharacterized genes by employing the invention to reduce the amount and/or alter the timing of target gene activity. The invention could be used in determining potential targets for pharmaceuticals, understanding normal and pathological events associated with development, determining signaling pathways responsible for postnatal development/aging, and the like. The increasing speed of acquiring nucleotide sequence information from genomic and expressed gene sources, including total sequences for mammalian genomes, can be coupled with the invention to determine gene function in a cell or in a whole organism. The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects. Expression of a shRNA or other construct from a regulated polIII promoter provides additional flexibility to such functional genomics approaches, permitting temporal and spatial control as desired.

A simple assay would be to inhibit gene expression according to the partial sequence available from an expressed sequence tag (EST). Functional alterations in growth, development, metabolism, disease resistance, or other biological processes would be indicative of the normal role of the EST's gene product. By using a regulated promoter, the effect of silencing of the target gene at different developmental or other timepoints could be assessed. Different levels of silencing may also be investigated.

The ease with which the dsRNA construct can be introduced into an intact cell/organism containing the target gene allows the present invention to be used in high throughput screening (HTS). For example, duplex RNA can be produced by an amplification reaction using primers flanking the inserts of any gene library derived from the target cell or organism. Inserts may be derived from genomic DNA or mRNA (e.g., cDNA and cRNA). Individual clones from the library can be replicated and then isolated in separate reactions, but preferably the library is maintained in individual reaction vessels (e.g., a 96 well microtiter plate) to minimize the number of steps required to practice the invention and to allow automation of the process.

In certain aspects, the invention provides methods for evaluating gene function in vivo. A cell containing a regulated shRNA expression construct designed to decrease expression of a target gene may be introduced into an animal and a phenotype may be assessed to determine the effect of the decreased gene expression. An entire animal may be generated from cells (e.g., ES cells) containing an shRNA expression construct designed to decrease expression of a target gene. A phenotype of the transgenic animal may be assessed.

The animal may be essentially any experimentally tractable animal, such as a non-human primate, a rodent (e.g., a mouse), a lagomorph (e.g., a rabbit), a canid (e.g. a domestic dog), a feline (e.g., a domestic cat). In general, animals with complete or near complete genome projects are preferred.

A phenotype to be assessed may be anything of interest. Quantitating the tendency of a stem cell to contribute to a particular tissue or tumor is a powerful method for identifying target genes that participate in stem cell differentiation and in tumorigenic and tumor maintenance processes. Phenotypes that have relevance to a disease state may be observed, such as susceptibility to a viral, bacterial or other infection, insulin production or glucose homeostasis, muscle function, neural regeneration, production of one or more metabolites, behavior patterns, inflammation, production of autoantibodies, obesity, etc.

A panel of shRNAs that affect target gene expression by varying degrees may be used, and phenotypes may be assessed. In particular, it may be useful to measure any correlation between the degree of gene expression decrease and a particular phenotype. A regulated promoter that may be activated to differing degrees may also be used to assess the effect of quantitatively different levels of silencing.

A heterogeneous pool of regulated shRNA constructs may be introduced into cells, and these cells may be introduced into an animal. In an embodiment of this type of experiment, the cells will be subjected to a selective pressure and then it will be possible to identify which shRNAs confer resistance or sensitivity to the selective pressure. The selective pressure may be quite subtle or unintentional, for example, mere engraftment of transfected HSCs may be a selective pressure, with some shRNAs interfering with engraftment and others promoting engraftment. Development and differentiation may be viewed as a “selective pressure”, with some shRNAs modulating the tendency of certain stem cells to differentiate into different subsets of progeny. Treatment with a chemotherapeutic agent may be used as selective pressure, as described below. The heterogeneous pool of shRNAs may be obtained from a library, and in certain preferred embodiments, the library is a barcoded library, permitting rapid identification of shRNA species.

In certain aspects, the invention provides methods for identifying genes that affect the sensitivity of tumor cells to a chemotherapeutic agent. The molecular mechanisms that underlie chemoresistance in human cancers remain largely unknown. While various anticancer agents clearly have different mechanisms of action, most ultimately either interfere with DNA synthesis or produce DNA damage. This, in turn, triggers cellular checkpoints that either arrest cell proliferation to allow repair or provoke permanent exit from the cell cycle by apoptosis or senescence.

In certain embodiments, a method comprises introducing into a subject a transfected stem cell comprising a nucleic acid construct encoding a regulated shRNA, wherein the shRNA is complementary to at least a portion of a target gene, wherein the transfected stem cell exhibits decreased expression of the target gene, and wherein the transfected stem cell gives rise to a transfected tumor cell in vivo. For example, the stem cell may be derived from an animal that has a genetic predisposition to tumorigenesis, such as an oncogene over-expressing animal (e.g. Eμ-myc mice) or a tumor suppressor knockout (e.g., p53 −/− animal). Alternatively, an animal comprising the stem cells may be exposed to carcinogenic conditions such that tumors comprising cells derived from the stem cells are generated. An animal having tumors may be treated with a chemotherapeutic or other anti-tumor regimen, and the effect of this regimen on cells expressing the shRNA may be evaluated. An shRNA that is overrepresented following anti-tumor therapy is likely to be targeted against a gene that confers sensitivity. An shRNA that is underrepresented following anti-tumor therapy is likely to be targeted against a gene that confers resistance. An shRNA that is underrepresented may be developed for use as a co-therapeutic to be co-administered with the chemotherapeutic agent in question and suppress resistance.

Overrepresentation and underrepresentation are generally comparative terms, and determination of these parameters will generally involve comparison to a control or benchmark. A comparison may simply be to the same animal prior to chemotherapy administration. A comparison may also be to a control subject that has not received the chemotherapeutic agent. A comparison may be to an average of multiple other shRNA trials. Any control need not be contemporaneous with the experiment, although the protocol should be substantially the same.

This technique may be performed on individual shRNAs. The technique may also be adopted for highly parallel screening. For example, a method may comprise introducing into a subject a plurality of transfected stem cells, wherein each transfected stem cell comprises a nucleic acid construct comprising a representative shRNA of an shRNA library, and wherein a representative shRNA of an shRNA library is complementary to at least a portion of a representative target gene, wherein a plurality of the transfected stem cells exhibits decreased expression of a representative target gene, and wherein a plurality of the transfected stem cells gives rise to transfected tumor cells in vivo. Notably, it is not necessary or expected that every shRNA is different or that every transfected cell will become part of a tumor. Once tumors have been generated, a chemotherapeutic or other anti-tumor regimen may be administered, and the overrepresentation or underrepresentation of shRNA species may be evaluated. In certain preferred embodiments, each representative shRNA is associated with a distinguishable tag that permits rapid identification of each shRNA. For example, shRNAs may be obtained from a shRNA library that is barcoded.

Certain methods described herein take advantage of the fact that large numbers of cancer cells (e.g., lymphoma cells) can be isolated from affected mice and transplanted into syngeneic, immunocompetent recipients to create a lymphoma that is virtually indistinguishable from the spontaneous disease. This allows in vitro manipulation of tumor cells to create potentially chemoresistant variants that can be analyzed in vivo. In certain exemplary embodiments, the invention exploits advantages of the Eμ-myc system to undertake an unbiased search for genetic alterations that can confer resistance to chemotherapeutics, such as the widely used alkylating agent, CTX.

The following is an outline of an example of a screen to identify genes that confer resistance to CTX using an unbiased, genetic approach. Populations of isolated lymphoma cells from the Eμ-myc mouse receive pools of sequence verified shRNAs that specifically target murine genes. Engineered cells are introduced into immunocompetent, syngeneic recipient animals. Upon the appearance of tumors, the animals are be treated with CTX. In each case, the time of remission is measured, and, upon relapse, the animals undergo a second round of treatment. After two rounds of therapy, the shRNA resident in resistant populations are identified and transferred into fresh populations of lymphoma cells, which are transplanted into naïve animals. After the appropriate number of selection cycles, individual shRNAs that are capable of conferring drug resistance are obtained.

In certain embodiments, a regulated expression construct that transcribes an RNAi species, e.g., a dsRNA or hairpin RNA, can include a barcode sequence. For those embodiments in which the RNAi constructs are provided as a variegated library for generating different RNAi species against a variety of different target sequence, each member (e.g., each unique target sequence) of the library can include a distinct barcode sequence such that that member of the library can be later identified if isolated individually or as part of an enriched population of RNAi constructs.

For example, two methods for determining the identity of the barcode sequence are by chemical cleavage, as disclosed by Maxim and Gilbert (1977), and by chain extension using ddNTPs, as disclosed by Sanger et al. (1977). In other embodiments, the sequence can be obtained by techniques utilizing capillary gel electrophoresis or mass spectroscopy. See, for example, U.S. Pat. No. 5,003,059.

Alternatively, another method for determining the identity of a barcode sequence is to individually synthesize probes representing each possible sequence for each character position of a barcode sequence set. Thus, the entire set would comprise every possible sequence within the barcode sequence portion or some smaller portion of the set. By various deconvolution techniques, the identity of the probes which specifically anneal to the barcode sequence sequences can be determined. An exemplary procedure would be to synthesize one or more sets of nucleic acid probes for detecting barcode sequence sequences simultaneously on a solid support. Preferred examples of a solid support include a plastic, a ceramic, a metal, a resin, a gel, and a membrane. A more preferred embodiment comprises a two-dimensional or three-dimensional matrix, such as a gel, with multiple probe binding sites, such as a hybridization chip as described by Pevzner et al. (J. Biomol. Struc. & Dyn. 9: 399-410, 1991), and by Maskos and Southern (Nuc. Acids Res. 20: 1679-84, 1992).

Hybridization chips can be used to construct very large probe arrays which are subsequently hybridized with a target nucleic acid. Analysis of the hybridization pattern of the chip provides an immediate fingerprint identification of the barcode sequence sequence. Patterns can be manually or computer analyzed, but it is clear that positional sequencing by hybridization lends itself to computer analysis and automation. Algorithms and software have been developed for sequence reconstruction which are applicable to the methods described herein (Drmanac et al., (1992) Electrophoresis 13: 566-73; P. A. Pevzner, J. Biomol. Struc. & Dyn. 7: 63-73, 1989).

For example, the identity of the barcode sequence sequence can be determined by annealing a solution of test sample nucleic acid including one or more barcode sequence sequences to a fixed array of character detection oligonucleotides (barcode sequence probes), where each column in the array preferably codes for one character of the barcode sequence. Each fixed oligonucleotide has a nucleotide base sequence that is complementary to the nucleotide base sequence of a single character. Either the test sample nucleic acid or the fixed oligonucleotides can be labeled in such a fashion to permit read-out upon hybridization, e.g., by radioactive labeling or chemiluminescent labeling. Test nucleic acid can be labeled, for example, by using PCR to amplify the identification region of a DNA pool under test with PCR primers that are radioactive or chemiluminescent. Preferred detectable labels include a radioisotope, a stable isotope, an enzyme, a fluorescent chemical, a luminescent chemical, a chromatic chemical, a metal, an electric charge, or a spatial structure. There are many procedures whereby one of ordinary skill can incorporate detectable label into a nucleic acid.

For example, enzymes used in molecular biology will incorporate radioisotope labeled substrate into nucleic acid. These include polymerases, kinases, and transferases. The labeling isotope is preferably, ³²P, ³⁵S, ¹⁴C, or ¹²⁵I.

Other, more advanced methods of detection include evanescent wave detection of surface plasmon resonance of thin metal film labels such as gold, by, for example, the BIAcore sensor sold by Pharmacia, or other suitable biosensors. An exemplary plasmon resonance technique utilizes a glass slide having a first side on which is a thin metal film (known in the art as a sensor chip), a prism, a source of monochromatic and polarized light, a photodetector array, and an analyte channel that directs a medium suspected of containing an analyte, in this case a barcode sequence-containing nucleic acid, to the exposed surface of the metal film. A face of the prism is separated from the second side of the glass slide (the side opposite the metal film) by a thin film of refractive index matching fluid. Light from the light source is directed through the prism, the film of refractive index matching fluid, and the glass slide so as to strike the metal film at an angle at which total internal reflection of the light results, and an evanescent field is therefore caused to extend from the prism into the metal film. This evanescent field can couple to an electromagnetic surface wave (a surface plasmon) at the metal film, causing surface plasmon resonance. When an array of barcode sequence probes are attached to the sensor chip, the pattern of annealing to barcode sequence sequences produces a detectable pattern of surface plasmon resonance on the chip.

The pattern of annealing, e.g., of selective hybriziation, of the labeled test DNA to the oligonucleotide array or the test DNA to the labeled oligonucleotide array permits the barcode sequence present in the original DNA clone to be directly read out. The detection array can include redundant oligonucleotides to provide integrated error checking.

In general, the hybridization will be carried out under conditions wherein there is little background (non-specific) hybridization, e.g., the background level is at least one order of magnitude less than specific binding, and even more preferably, at least two, three or four orders of magnitude less.

Additionally, the array can contain oligonucleotides that are known not to match any barcode sequence in the library as a negative control, and/or oligonucleotides that are known to match all barcode sequences, e.g., primer flanking sequence, as a positive control.

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999.

Exemplification

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other stem cell sources and selection methods, other culture media and culture methods, other dosage and treatment schedules, and other animals and/or humans, all without limitation, can be employed, without departing from the scope of the invention as claimed.

The following experimental procedures were used in performing the examples below.

### Materials and Methods

Generation of retroviral constructs. Retroviral vectors expressing the two nuclear receptors/transcription factors VgEcR and RXR were constructed as described previously (17). To construct pEind-RNAi, a GAL4-Oct-2^{Q}(Q→A) fragment was PCR amplified from pCG-GAL4-Oct-2^{Q}(Q→A) (18) using a forward primer (ACGCCCGCGGATGAA GCTACTGTCTTCTATC) and reverse primer (CACCCTGAAGTTCTCAGGATCC), digested with Sac II/BamH I and inserted into the Sac II/Bam H1 site upstream of vector pIRES-EGFP [Clontech, Palo Alto, Calif.]. Next the GAL4-Oct-2^{Q}(Q→A) IRES-linked EGFP was PCR amplified using a forward primer (AGCTTTGTTTAAACCGAATTCTGCAGTCGACGGTA) and reverse primer CAGCTGATCATTA CTTGTACAGCTCGTCC, digested with Pme I/Bcl I and inserted into the Pme I/Bcl I site of pI-TKHygro retroviral vector (17). Next, a Bgl II site was created downstream of Xho I in pI-TKHygro by site directed mutagenesis (Stratagene, CA) using two primers ACAGTGGCGGCCGCTCGAGATCTCTTGGAGTGGTG AATCCGTT (upper) and TGTCACCGCCGGCGAGCTCTAG AGAACCTCACCACTTAGG CAA (lower). The resulting construct was digested with Bgl II and blunt ended with Klenow enzyme into which, a gateway destination cassette ccdB (Invitrogen, CA) was inserted. To generate 4XGAL4 DNA binding sites upstream of the U6 promoter-containing vector (19), a Sac I site was created by site directed mutagenesis (Stratagene, CA) upstream of Oct-1 binding site using two primers CAGGCTCCGCGGCCGCCGAGCTCACCGAGGG CCTATTTCCCATG (upper) and GTCCGAGGCGCCG GC GGCTCGAGTGGCTC CCGGATAAAGGGTAC (lower). The Oct-1 and staf binding elements were removed by digestion with SacI/NdeI and replaced with 4XGAL4 binding sites obtained by digesting vector pU6/-198-4XG17M (18) with Sac I/Nde I.

Designing and cloning of shRNAs. A majority of shRNA probes were designed using computer software (http://www.cshl.org/public/SCIENCE/hannon.html). shRNA sequences (two complementary ˜83 nt DNA oligos) were annealed and cloned directly into a 4XGAL4 U6 promoter-containing vector using a ligation-independent cloning method (19). The entire 4XGAL4 U6 promoter-shRNA cassette was transferred into pEind-RNAi by gateway clonase recombination reaction (Invitrogen). shRNA against the p53 gene was designed based on a published sequence (11). shRNA against firefly luciferase (9) served as a non-specific control.

Cell-culture, retroviral transductions and selection of stable cells. U87MG (human glioblastoma derived cells, ATTC no. HTB-14) was maintained in DMEM supplemented with 10% fetal bovine serum (FBS), 0.1 mg/ml penicillin and 0.1 mg/ml streptomycin (Life technologies, Rockville, Md.). The murine endothelial cell line, mHEVc was grown in RPMI medium supplemented with 10% fetal bovine serum (FBS), 1 mM HEPES/10 mM sodium bicarbonate and 2 mM glutamine. U87MG stably expressing receptors VgEcR and RXR (17), was maintained in puromycin (0.4 mg/ml) and G418 (1 μg/ml) selection, whereas mHEVc was maintained in puromycin (0.6 mg/ml) and G418 (6 μg/ml). Both of the host cell lines carrying pEind-RNAi were maintained in hygromycin at 0.1 mg/ml. For biosafety purposes the pEind-RNAi is a self-inactivating (sin) retrovirus that lacks U3 enhancers in the 3′LTR. On proviral integration, this deletion flanks the retroviral insert thereby removing the enhancers from both 5′ and 3′ LTRs minimizing the risk of generating replication competent recombinants.

High titer virus were produced by calcium phosphate transfection of retroviral DNA constructs into the LinX amphotrophic retroviral packaging cell line (17) followed by incubation with dexamethasone/butyrate (1 μM/ml) of packagers at 32° C. for 3 days. Infections were carried out by harvesting supernatants from LinX packagers, filtered through a 0.45 μM membrane (Millipore), followed by the addition of polybrene (8 μM) to supernatants, finally overlaying onto actively dividing host cells (70% confluent). Cells were gently spun at room temperature for 30 min, incubated at 32° C. for 6 h. Recipients were infected one more time using freshly prepared supernatant from the same packaging plate, at 8 h interval. At 24 h after the final infection, cells were split to lower densities and antibiotic selection applied for 2-3 days. Cells were split on 6 well plates and induced with 2 μM murA and the top 3% GFP⁺ cells were collected by FACS. Cells that expressed EGFP in the absence of inducer, were removed by FACS. The GFP⁺ cells were expanded for further analysis. An equal amount of ethanol was added to the plates that were uninduced.

Western blot analysis and antibodies. Stable cell lines inducibly expressing shRNAs targeting human p53 or murine MyoD mRNA were separately grown in 6 well plate at a density of 0.3×105/well. Cells were induced with the indicated concentrations of murA and 72 h post-induction cells were harvested and lysed with RIPA lysis buffer (150 mM NaCl, 50 mM Tris, pH 8.0, 0.5% sodium deoxycholate, 0.1% SDS and 1% NP40) containing complete protease inhibitor (Roche Applied Science, Germany). Equal amounts of lysate were subjected to Western blot analysis (20) using antibodies against p53 (1:1000, Novocastra Laboratories Ltd.), MyoD (1:500), GAL4 (1:1000), p21 (1:500), EGFP (1:200) and HRPO-conjugated secondary antibody (1:5000), obtained from Santa Cruz Biotechnology. The blots were stripped by incubating with 100 mM β-mercaptoethanol, 2% SDS, 62.5 mM Tris-HCl (pH 6.7) at 50° C. for 20 min and reprobed with anti β-tubulin (1:5000, Sigma) primary antibody and HRPO-conjugated secondary antibody (1:3000, Amersham Biosciences, NJ) to show equal loading. The blots were developed using ECL Plus (Amersham, Biosciences, NJ) or West Femto (Pierce) blotting detection system. Signal intensities were determined after background corrections by using Alpha-Imager 2000 Documentation and Analysis Software (Alpha Innotech, San Leandro, Calif.). The percentage reduction in band intensity for each concentration of murA was calculated relative to the uninduced samples and normalized against β-tubulin.

Northern-blot analysis. Cells were plated at a density of 1×105 in a 10 cm dish and induced with 5 μM murA. After 72 h cells were harvested and total RNA was extracted using a RNeasy kit (Qiagen). 30 μg RNA was loaded on a 15% denaturing polyacrylamide-urea gel and northern blot analysis was performed (21). 21-nt sense strand of p53 (GACTCCAGTGGTAATCTACTT) was end labeled with γ-³²P-ATP using a KinaseMax kit (Ambion). Hybridization was performed using a NorthernMax kit (Ambion). Post-hybridization, blots were washed and exposed to X-ray film at −70° C.

Immunofluoresence. U87MG cells stably and inducibly expressing human p53 shRNA or non-specific shRNA were separately grown on glass cover slips in a 12-well plate at a density of 1×103 cells/well. Non-specific shRNA was targeted against Luciferase gene. Cells were induced with 5 μM murA for 72 h, fixed in 4% paraformaldehyde for 30 min and washed 3 times for 5 min with PBS, followed by permeabilization in 0.2% Triton-X-100 and 0.5% normal goat serum for 10 min. The permeabilized cells were blocked for 30 min in 10% normal goat serum and incubated with p53 antibody (1:200, Novocastra Laboratories Ltd.) and β-tubulin (1:400, Sigma) for 1 h at room temp, and washed 3 times for 5 min with PBS and 0.5% normal goat serum. Next, cells were incubated with secondary antibodies, Alexa 488 and Alexa 594 (Molecular probes, Oregon), for 1 h at room temp and stained with DAPI (1 μg/ml). Cover slip was mounted on antifade mounting media (Fluoromount-G, Southern Biotechnology Associates) and visualized under a fluorescent microscope. Images were captured using a Zeiss AxioCam HRm camera at equal exposure time for all panels.

Flow cytometric analysis of cell cycle distribution. U87MG cells stably and inducibly expressing human p53 shRNA were plated at a density of 1×105 cells/well in 6-well plate and induced with 5 μM murA for 72 h followed by γ-irradiation at 20 Gy. In reversal experiments cells were trypsinized and replated to maintain appropriate densities. 24 h after γ-irradiation, adherent cells were harvested, washed once in phosphate-buffered saline (PBS), and fixed in ice-cold 70% ethanol in distilled water. Cells were then washed twice in PBS supplemented with 1% BSA and resuspended in PBS containing 0.1% Triton X-100, 50 μg per ml of propidium iodide, 5 mM sodium citrate and 50 μg per ml of RNase A. After incubation at room temp for 20 min, cells were analyzed for cell cycle distribution with an LSRII flow cytometer (Becton Dickinson) and FACSDiva software (Becton Dickinson). Red fluorescence (585 V 42 nm) was evaluated on a linear scale, and pulse width analysis was used to exclude cell doublets and aggregates from the analysis. Cells with DNA content between 2N and 4N were designated as being in the G1, S, or G2/M phase of the cell cycle. The number of cells in each compartment of the cell cycle was expressed as a percentage of the total number of cells present.

### EXAMPLE 1

### Design and Characterization of the Retroviral Based Ecdysone Inducible RNAi System

The U6 promoter is widely used for directing expression of shRNAs because it is active in all cell types and efficiently directs synthesis of small, non-coding transcripts bearing well-defined ends. However, so far, attempts to generate robust inducible pol III promoters have met with less than satisfactory results (22). To facilitate stable and inducible suppression of any gene we developed an ecdysone-inducible synthesis of short hairpin RNAs (shRNAs) under the control of a modified U6 promoter. We accomplished this by replacing the natural U6 enhancers with heterologous GAL4-DNA binding sites and tested GAL4-transactivator fusions (18), that have been shown before to activate transcription specifically from the wild-type U6 promoter, but not from pol II mRNA, U1 snRNA, or U6 TATA-promoters. Among those, the synthetic transactivator, Oct-2^{Q}(Q→A) specifically expressed shRNA with no background expression in the absence of the inducer, and was, therefore, used for further analysis.

The ecdysone inducible system is comprised of three Moloney murine leukemia virus (MoMLV)-based retroviral vectors, the two vectors expressing nuclear receptors/transcription factors VgEcR and RXR, and a third construct, pEind-RNAi expressing a chimeric transactivator GAL4-Oct-2^{Q}(Q→A), and an internal ribosomal entry site (IRES) linked enhanced green fluorescence protein (EGFP) under an inducible promoter E/GRE/Hsmin (FIG. 1A). GFP expression permits enrichment of transduced cells by fluorescence-activated cell sorting (FACS). We incorporated a “gateway” site-specific acceptor, ccdB (Invitrogen), so that any DNA encoded shRNA of interest can be readily transferred from a donor vector by recombination downstream of the U6 promoter. The enhancer of the U6 promoter is comprised of an octamer motif and an adjacent element that binds the transactivators Oct-1 and staf respectively (23). We deleted the natural enhancer region and engineered four tandem GAL4 binding sites (4XGAL4) in its place. Upon induction with muristerone A (murA, an analogue of ecdysone) the two receptors/transcription factors dimerize and bind to the hybrid ecdysone responsive element (E/GRE) to activate GAL4-Oct-2^{Q}(Q→A) expression. GAL4-Oct-2^{Q}(Q→A) in turn binds to the 4XGAL4 DNA binding sites and activates the U6 promoter, which drives expression of a shRNA (FIG. 1A).

Stable cell lines were generated as described in the Experimental Protocols (also see Supplementary FIG. 1). The use of a retroviral delivery and flow sorting of EGFP⁺ cells expedited rapid and efficient generation of stable cell lines both in murine and human cells. Analysis of sorted cells following induction with murA showed >95% GFP⁺ cells as determined by fluorescent microscopy (FIG. 1B), and FACS (FIG. 1C). Western blot analysis demonstrated increased levels of GAL4-Oct-2^{Q}(Q→A) and EGFP protein in samples treated with murA but not in untreated controls (FIG. 1D). Having established inducible expression of the activator GAL4-Oct-2^{Q}(Q→A), we next demonstrated its ability to activate p53 shRNA expression from the modified U6 promoter. Northern blot analysis showed production of 21-nt siRNAs specifically in the cells expressing the activator (FIG. 1E). These results indicate that the GAL4-Oct-2^{Q}(Q→A) induced formation of the stem-loop precursor transcript that was cleaved in the cell to produce a functional siRNA. Taken together, these data indicate that the induction is highly specific and lacks detectable background expression levels in the absence of the inducer.

### Stable and Efficient RNAi-Mediated Inducible Suppression of Human p53 Gene

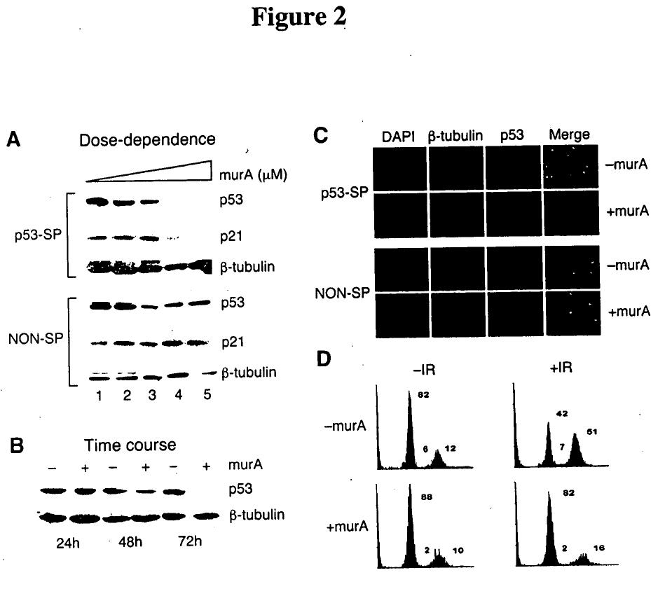

The usefulness of the RNAi-inducible system in suppressing expression of a cognate target gene was evaluated next. As a first target we chose the human p53 gene because of detectable expression in mammalian cells, availability of reliable antibodies to monitor levels of the protein, and the presence of an effective shRNA against the p53 gene (11). A human glioblastoma cell line, U87MG carrying a functional wild-type p53 (24), and stably expressing receptors, VgEcR and RXR was transduced with a pEind-RNAi virus expressing an shRNA targeting the p53 gene. Stable cells treated with 0.5 μM to 5 μM murA showed a dose-dependent reduction in endogenous p53 level with almost 95% reduction in the presence of 5 μM murA (FIG. 2A; upper panel). The suppression of p53 was also time-dependent showing partial reduction (60%) at 48 h and maximal reduction (>95%) at 72 h post-induction (FIG. 2B). To ascertain that the induced shRNA targeted the p53 mRNA specifically we measured the levels of cyclin-dependent kinase inhibitor p21, a well characterized transcriptional target of p53. As expected, the decrease in p21 levels correlated well with inducible suppression of p53 (FIG. 2A; upper panel), relative to that obtained with a non-specific shRNA (FIG. 2A; lower panel). In contrast, levels of Cdk4 protein, an upstream component of the p53-signaling pathway remained unchanged (data not shown). Taken together, these results suggest that the p53 shRNA synthesized by the inducible system is tightly regulated and exhibits high target-specificity.

We next assessed the effects of inducing expression of p53 shRNA at a single cell level by immunofluorescence experiments. The results showed the presence of nuclear p53 in the absence of induction (FIG. 2C; upper panel, top), as expected. Induction of p53 shRNA expression resulted in a dramatic reduction in the levels of p53 (FIG. 2C; upper panel, bottom), relative to that obtained with a non-specific shRNA (FIG. 2C; lower panel). Strikingly, cells with reduced levels of p53 were flat and large in contrast to normal long spindle forms observed in the uninduced state (FIG. 2C; upper panel). The morphological change was specific to effects of p53 hairpin and not to the activity of either the inducer or the expression of GAL4-Oct-2^{Q}(Q→A), EGFP and non-specific shRNA (FIG. 2C; lower panel). Although reports indicating morphology changes in HeLa cells as a consequence of p53 suppression by antisense RNA are available (25), it is likely that in the U87MG cells suppression of p53 cooperates with absence of PTEN (26, 27), to generate the observed phenotype.

After having established specific downregulation of p53 gene expression by inducible RNAi, we next wondered if the p53 suppression had a functional consequence. FACS analysis showed a dramatic increase in the cell number in G2/M-phase (39%) and a concomitant decrease in G0/G I-phase (40%) in uninduced, irradiated samples (FIG. 2D; upper panel, right). In contrast, cells induced for p53 shRNA and exposed to γ-irradiation lost their ability to arrest at the G2/M phase of the cell cycle (FIG. 2D; lower panel, right). We were unable to observe G0/G1 arrest in multiple independent experiments, underscoring the importance of p53 in mediating G2/M arrest in response to DNA damage in U87MG cells consistent with published observations (28). Indeed, cell cycle arrest in G2/M has been shown to depend on the cell type (29, 30).

### The RNAi-Mediated Inducible Gene Suppression is Reversible

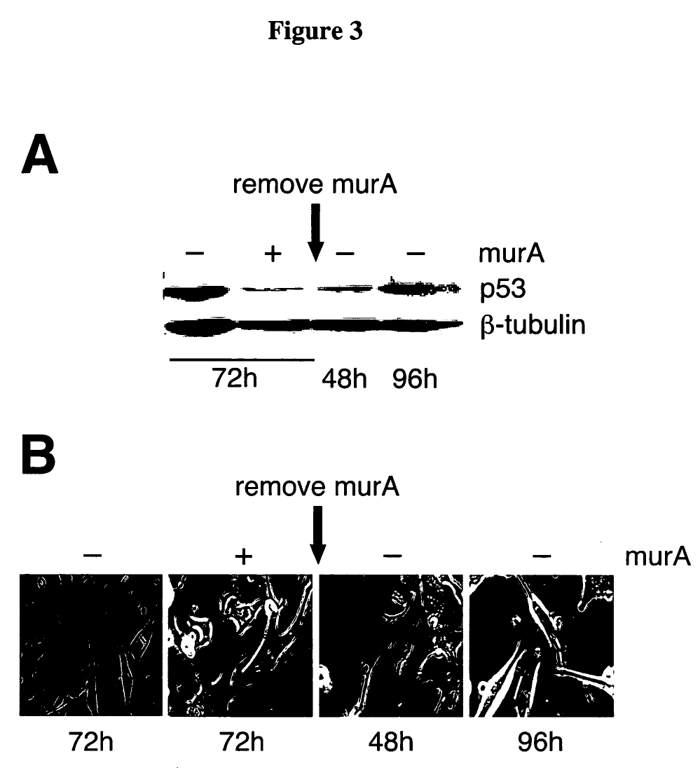

A major limitation of constitutive shRNA expression systems is the irreversible suppression of gene expression that could result in non-physiological responses. We determined whether the gene suppression obtained by the RNAi inducible system could be reversed after withdrawal of the inducer. In the presence of the inducer stable cells carrying p53 shRNA showed, as expected, reduction in endogenous p53 levels (FIG. 3A), and associated morphological changes at 72 h post-induction (FIG. 3B). Upon removal of the inducer there was a partial recovery in p53 protein levels at 48 h with almost full recovery at 96 h (FIG. 3A). Noticeably, the recovery of p53 protein level was associated with restoration in the original cell morphology (FIG. 3B, compare large flat cells at 72 h in the presence of murA to spindle shaped cells at 96 h following withdrawal of murA), and cell function as determined by γ-irradiation induced cell cycle arrest (see Supplementary FIG. 2), underscoring the swift clearance kinetics of murA that results in a rapid phenotypic switch. These studies also demonstrate that the p53 gene suppression does not lead to an irreversible cascade of molecular events.

### Inducible Suppression of MyoD in a Murine Cell Line

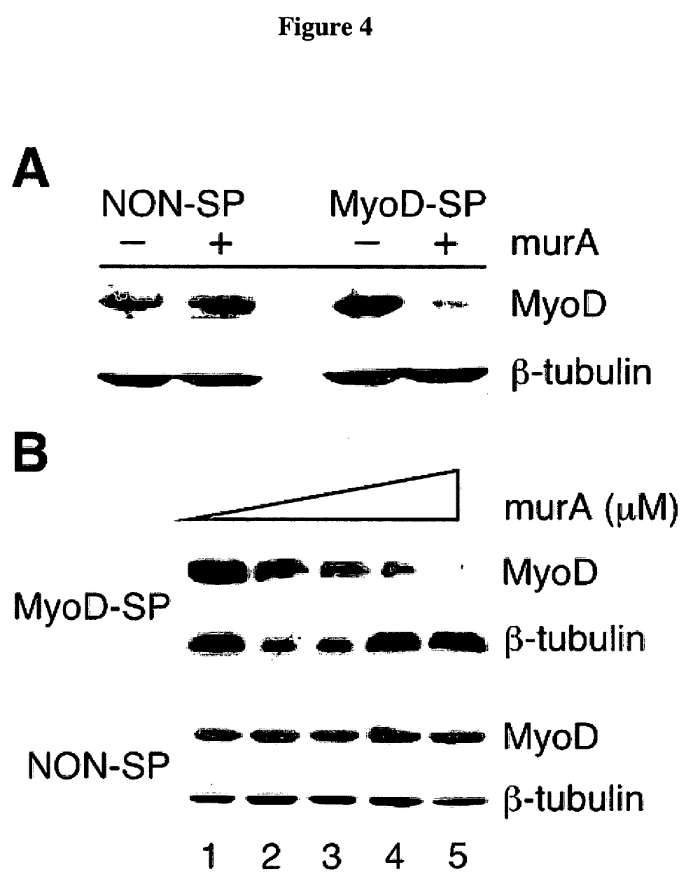

The general utility of the RNAi-inducible system was determined by targeting suppression of a MyoD gene in a murine cell line. Murine endothelial cells, mHEVc (31), were transduced with viruses carrying the two receptors/transcription factors and pEind-RNAi expressing a MyoD shRNA, as described earlier. Efficient suppression of MyoD gene expression was observed at 72 h post-induction (FIG. 4A). Furthermore, increasing concentrations of murA (0.5 μM to 5 μM) showed a dose-dependent reduction in endogenous MyoD levels with almost 95% reduction in the presence of 5 μM murA (FIG. 4B; upper panel), relative to cells stably expressing a non-specific shRNA (NON-SP, FIG. 4B; lower panel).

In summary, we provide a powerful new approach to stably and inducibly suppress gene expression in mammalian cells. Given the success of the ecdysone-based inducible systems in animal models (15, 32), and germ line transmission of RNAi (33), it is conceivable to generate transgenic animals inducibly expressing RNAi. A considerable improvement to the existing design would be to obtain tissue-specific regulation in vivo by either expressing the GAL4-activator under the control of a tissue-specific promoters or by utilizing a “caged ecdysteroid” (34). Recently, a lentiviral meditated doxycyclin-controllable conditional suppression of genes in mammalian cells was reported (35). However, it was not clear how much basal expression in the absence of the inducer the system allows. Furthermore, doxycyclin is linked to toxicity problems, a major disadvantage for studies with both cultured cells and animals. Ecdysone is more suitable for use in vivo because it is a naturally occurring lipophilic steroid that can penetrate tissues and is quickly metabolized and cleared (32).

A major application of the RNAi-inducible system would be in studies where partial down-regulation of gene expression is desired, particularly in cases where partial suppression result in distinct phenotypes. For example, shRNAs showing varying levels of p53 suppression generated distinct tumor phenotypes in vivo (36). Partial suppression is also useful where lethality associated with complete suppression of essential genes is of concern. In our system, the levels of shRNA expression can be easily and finely controlled by simply varying the dosage of the inducer.

### REFERENCES

1. Hammond, S. M., Boettcher, S., Caudy, A. A., Kobayashi, R. & Hannon, G. J. (2001) Science 293, 1146-50.
2. Sharp, P. A. (1999) Genes Dev 13, 139-41.
3. Elbashir, S. M., Harborth, J., Lendeckel, W., Yalcin, A., Weber, K. & Tuschl, T. (2001) Nature 411, 494-8.
4. Bernstein, E., Caudy, A. A., Hammond, S. M. & Hannon, G. J. (2001) Nature 409, 363-6.
5. Paddison, P. J. & Hannon, G. J. (2002) Cancer Cell 2, 17-23.
6. Marcus, P. 1. & Sekellick, M. J. (2001) J Interferon Cytokine Res 21,423-9.
7. Gil, J. & Esteban, M. (2000) Apoptosis 5, 107-14.
8. Elbashir, S. M., Harborth, J., Weber, K. & Tuschl, T. (2002) Methods 26, 199-213.
9. Paddison, P. J., Caudy, A. A., Bernstein, E., Hannon, G. J. & Conklin, D. S. (2002) Genes Dev 16, 948-58.
10. Sui, G., Soohoo, C., Affar el, B., Gay, F., Shi, Y. & Forrester, W. C. (2002) Proc Natl Acad Sci USA 99, 5515-20.
11. Brummelkamp, T. R., Bernards, R. & Agami, R. (2002) Science 296, 550-3.
12. Paul, C. P., Good, P. D., Winer, 1. & Engelke, D. R. (2002) Nat Biotechnol 20, 505-8.
13. Rubinson, D. A., Dillon, C. P., Kwiatkowski, A. V., Sievers, C., Yang, L., Kopinja, J., Zhang, M., McManus, M. T., Gertler, F. B., Scott, M. L. & Van Parijs, L. (2003) Nat Genet 33, 401-6.
14. Gossen, M., Freundlieb, S., Bender, G., Muller, G., Hillen, W. & Bujard, H. (1995) Science 268, 1766-9.
15. No, D., Yao, T. P. & Evans, R. M. (1996) Proc Natl Acad Sci USA 93, 3346-51.
16. Van Craenenbroeck, K., Vanhoenacker, P., Leysen, J. E. & Haegeman, G. (2001) Eur J Neurosci 14, 968-76.
17. Stolarov, J., Chang, K., Reiner, A., Rodgers, L., Hannon, G. J., Wigler, M. H. & Mittal, V. (2001) Proc Natl Acad Sci USA 98, 13043-8.
18. Das, G., Hinkley, C. S. & Herr, W. (1995) Nature 374, 657-60.
19. Kumar, R., Conklin, D. S. & Mittal, V. (in press) Genome Research.
20. Harlow, E. a. L., D. (1988) Cold Spring Harbor Laboratory Press, Cold Spring Harbor, N.Y.
21. Dalmay, T., Hamilton, A., Rudd, S., Angell, S. & Baulcombe, D. C. (2000) Cell 101, 543-53.
22. Meissner, W., Rothfels, H., Schafer, B. & Seifart, K. (2001) Nucleic Acids Res 29, 1672-82.
23. Schaub, M., Myslinski, E., Schuster, C., Krol, A. & Carbon, P. (1997) Embo J 16, 173-81.
24. Van Meir, E. G., Kikuchi, T., Tada, M., Li, H., Diserens, A. C., Wojcik, B. E., Huang, H. J., Friedmann, T., de Tribolet, N. & Cavenee, W. K. (1994) Cancer Res 54, 649-52.
25. Iotsova, V. & Stehelin, D. (1995) Eur J Cell Biol 68, 122-32.
26. Mayo, L. D., Dixon, J. E., Durden, D. L., Tonks, N. K. & Donner, D. B. (2002) J Biol Chem 277, 5484-9.
27. Stambolic, V., MacPherson, D., Sas, D., Lin, Y., Snow, B., Jang, Y., Benchimol, S. & Mak, T. W. (2001) Mol Cell 8, 317-25.
28. Badie, B., Goh, C. S., Klayer, J., Herweijer, H. & Boothman, D. A. (1999) Cancer Gene Ther 6, 155-62.
29. Taylor, W. R. & Stark, G. R. (2001) Oncogene 20, 1803-15.
30. Bunz, F., Dutriaux, A., Lengauer, C., Waldman, T., Zhou, S., Brown, J. P., Sedivy, J. M., Kinzler, K. W. & Vogelstein, B. (1998) Science 282, 1497-501.
31. Tudor, K. S., Deem, T. L. & Cook-Mills, J. M. (2000) Biochem Cell Biol 78, 99-113.
32. Karns, L. R., Kisielewski, A., Gulding, K. M., Seraj, J. M. & Theodorescu, D. (2001) BMC Biotechnol 1, 11.
33. Carmell, M. A., Zhang, L., Conklin, D. S., Hannon, G. J. & Rosenquist, T. A. (2003) Nat Struct Biol 10, 91-2.
34. Lin, W., Albanese, C., Pestell, R. G. & Lawrence, D. S. (2002) Chem Biol 9, 1347-53.
35. Wiznerowicz, M. & Trono, D. (2003) J Virol 77, 8957-61.
36. Hemann, M. T., Fridman, J. S., Zilfou, J. T., Hernando, E., Paddison, P. J., Cordon-Cardo, C., Hannon, G. J. & Lowe, S. W. (2003) Nat Genet 33, 396-400.
   Equivalents

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

Sequence Listings

SEQ ID NO:1 pU6-4XGAL4

1-8: Not I Restriction Site
10-15: Sac I Restriction Site
24-97: Four copies of Gal4 DNA binding sites
98-312: U6 Cassette
313-319: U6 transcription Termination

320-327: Not I site

GCGGCCGCCGAGCTCGGTACCCCGACGGAGTACTGTCCTCCGACGGAGTAC
TGTCCTCCGACGGAGTACTGTCCTCTGACGAGTAGTGTCCTCCGACGGGGAT
CCTCTAGAGTCATCGAGAGATAATTAGAATTAATTTGACTGTAAACACAAA
GATATTAGTACAAAATAGGTGACGTAGAAAGTAATAATTTCTTGGGTAGTTT
GCAGTTTTAAAATTATGTTTTAAAATGGACTATCATATGCTTACCGTAACTT
GAAAGTATTTCGATTTCTTGGCTTTATATATCTTGTGGAAAGGACGAAACAC
CGTTTTTTTGCGGCCGC

SEQ ID NO:2 pEind-RNAi
EGRE (5 E/GRE sites): 1362-1518
Start of transcription: 1591
Gal4 Oct2 Q>A (1878-2396) begins at ATG and terminates at TAG
IRES (2397-2994)
GFP (2995-3713) begins at ATG and terminates at TAA

ccdB: (3797-6067)

CTGCAGCCTGAATATGGGCCAAACAGGATATCTGTGGTAAGCAGTTCCT
GCCCCGGCTCAGGGCCAAGAACAGATGGAACAGCTGAATATGGGCCAA
ACAGGATATCTGTGGTAAGCAGTTCCTGCCCCGGCTCAGGGCCAAGAAC
AGATGGTCCCCAGATGCGGTCCAGCCCTCAGCAGTTTCTAGAGAACCAT
CAGATGTTTCCAGGGTGCCCCAAGGACGTGAAATGAGCCTGTGCCTTATT
TGAACTAACCAATCAGTTCGCTTCTCGCTTCTGTTCGCGCGCTTCTGCTCC
CCGAGCTCAATAAAAGAGCCCACAACCCCTCACTCGGGGCGCCAGTCCT
CCGATTGACTGAGTCGCCCGGGTAGCCGTGTATCCAATAAACCCTGTTGC
AGTTGCATCCGACTTGTGGTCTCGCTGTTCCTTGGGAGGGTCTCCTCTGA
GTGATTGACTACCGGTCAGCGGGGGTCTTTCATTTGGGGGCTCGTGGGGG
ATCGGGAGAGCCCTGGCCAGGGACCACCGACCCACCAGCGGAAGGCAA
GCTGGCCAGCAACTTATCTGTGTCTGTCCGATTGTCTAGTGTCTATGACT
GATTTTATGCGCCTGCGTCGGTACTAGTTAGCTAACTAGGTCTGTATCTG
GCGGACCCGTGGTGGAACTGACGAGTTCTGAACACCCGGCCGGAACCCT
GGGAGACGTCCCAGGGACTTTGGGGGCCGTTTTTGTGGCCCGAGCTGAG
GAAGGGAGTCGATGTGGAATCCGACCCCGTCAGGATATGTGGTTCTGGT
AGGAGACGAGAAGGTAAAACAGTTCCCGCCTCCGTCTGAATTTTTGCTTT
CGGTTTGGAACCGAAGCCGCGGGTCTTGTCTGCTGGAGCGCTGCAGCATC
GTTCTGTGTTGTCTCTGTCTGACTGTGTTTCTGTATTTGTCTGAAAATTAG
GGCCAGACTTGTACCACTCCCTTAAGTTTGACCTTAGGTCACTGGAAAGA
TGTCGAGCGGATCGCTCACAACCAGTCGGTAGATGTCAAGAAGAGACGT
TGGGTTACCTTCTGCTCTGCAGAATGGCCAACCTTTAACGTCGGATGGCG
GCGAGACGGCACCTTTAACCGAGACCTCATCACCCAGGTTAAGATCAAG
GTCTTTTCAGCTGGCCCGCATGGACAGCCAGACCAGGTCCCCTACATCGT
GACCTGGGAAGCCTTGGCTTTTGACCCCCCTCCCTGGGTGAAGCCCTTTG
TACACGCTAAGCCTCCGCCTCCTCTTCCTCCATCCGCCCCGTCTCTCCCCC
TTGAACCTCCTCGTTCGACCCCGCCTCGATCCTCCGTTTATCCAGCCCTCA
CTCCTTCTCTAGGCGCCGGCCGGATCTCGGCCGCATATTAAGTGCATTGT
TCTCGATACCGCTAAGTGCATTGTTCTCGTTAGCTCGATGGACAAGTGCA
TTGTTCTCTTGCTGAAAGCTCGATGGACAAGTGCATTGTTCTCTTGCTGA
AAGCTCGATGGACAAGTGCATTGTTCTCTTGCTGAAAGCTCAGTACCCGG
GAGTACCCTCGACCGCCGGAGTATAAATAGAGGCGCTTGGTCTACGGAG
CGACAATTCAATTCAAACAAGCAAAGTGAACACGTCGCTAAGCGAAAGC
TAAGCAAATAAACAAGCGCAGCTGAACAAGCTAAACAATCTGCAGTAA
AGTGCAAGTTAAAGTGAATCAATTAAAAGTAACCAGCAACCAAGTAAAT
CAACTGCAACTACTGAAATCTGCCAAGAAGTAATTATTGAATACAAGAA
GAGAACTCTGAATACTTTCAACAAGTTACCGAGAAAGAAGAACTCACAC
ACAGCTAGCGTTTAAACCGAATTCTGCAGTCGACGGTACCGCGATGAAG
CTACTGTCTTCTATCGAACAAGCATGCGATATTTGCCGACTTAAAAAGCT
CAAGTGCTCCAAAGAAAAAGCGAAGTGTGCCAAGTGTCTGAAGAACAAC
TGGGAGTGTCGCTACTCTCCCAAAACCAAAAGGTCTCCGCTGACTAGGG
CACATCTGACAGAAGTGGAATCAAGGCTAGAAAGACTGGAACAGCTATT
TCTACTGATTTTTCCTCGAGAAGACCTTGACATGATTTTGAAAATGGATT
CTTTACAGGATATAAAAGCATTGTTAACACTGCCAGGCTCTAGACCAAA
TCTATTCGCCCTACCTGCCGCTACCGCCGGAGCTCTTCTGACCTCCGCAC
CAAATCTATTGGCCCTACCTGCCGCTACCGCCGGAGCTCTTCTGACCTCC
GCACCAAATCTATTCGCCCTAGCTGCCGCTACGGCCGGAGCTCTTCTGAC
CTCCGCACGAAATCTATTCGCCCTACCTGCCGCTACCGCCGGAGCTCTTC
TGACCTCCGCACCATAGTCTTCGGATCCGCCCCTCTCCCTCCCCCCCCCCT
AACGTTACTGGCCGAAGCCGCTTGGAATAAGGCCGGNGTGCGTTTGTCT
ATATGTTATTTTCCACCATATTGCCGTCTTTTGGCAATGTGAGGGCCCGG
AAACCTGGCCCTGTCTTCTTGACGAGCATTCCTAGGGGTCTTTCCCCTCT
GGCCAAGGAATGCAAGGTCTGTTGAATGTCGTGAAGGAAGCAGTTCCTC
TGGAAGCTTCTTGAAGACAAACAACGTCTGTAGCGACCCTTTGCAGGCA
GCGGAACCCCCCACCTGGCGACAGGTGCCTCTGCGGCCAAAAGCCACGT
GTATAAGATACACCTGCAAAGGCGGCACAACCCCAGTGCCACGTTGTGA
GTTGGATAGTTGTGGAAAGAGTCAAATGGCTCTCCTCAAGCGTATTCAA
CAAGGGGCTGAAGGATGCCCAGAAGGTACCCCATTGTATGGGATCTGAT
CTGGGGCCTCGGTGCACATGCTTTAGATGTGTTTAGTCGAGGTTAAAAAA
ACGTCTAGGCCGCCCGAACCACGGGGACGTGGTTTTCCTTTGAAAAACA
CGATGATAATATGGCCACAACGATGGTGAGCAAGGGCGAGGAGCTGTTC
ACCGGGGTGGTGCCCATCCTGGTCGAGCTGGACGGGGACGTAAACGGCC
ACAAGTTCAGCGTGTCCGGGGAGGGCGAGGGCGATGGCACCTACGGCAA
GCTGACCCTGAAGTTCATCTGCACCACCGGCAAGGTGCCGGTGCCCTGGC
CCACCCTCGTGACCACCCTGACCTACGGCGTGCAGTGCTTCAGCCGCTAC
CCCGACCACATGAAGCAGGACGACTTCTTGAAGTCCGCCATGCCCGAAG
GCTACGTCCAGGAGCGCAGCATCTCTTCAAGGACGACGGCAAGTACAAG
ACCCGCGGCGAGGTGAAGTTGGAGGGCGAGACCCTGGTGAAGCGCATCG
AGCTGAAGGGCATCGACTTCAAGGAGGACGGCAAGATCCTGGGGGACA
AGCTGGAGTACAACTACAACAGCGACAACGTCTATATCATGGCCGACAA
GCAGAAGAACGGCATCAAGGTGAACTTGAAGATCCGCCACAACATCGAG
GACGGCAGCGTGCAGCTCGCCGACCACTACCAGCAGAACACCCCGATCG
GCGACGGCCCCGTGCTGCTGCCCGACAACCACTACCTGAGCAGCCAGTC
CGCCCTGAGCAAAGACCCCAAGGAGAAGCGCGATCACATGGTCCTGCTG
GAGTTCGTGACCGCCGCCGGGATCACTCTCGGCATGGACGAGCTGTACA
AGTAACTTAAGCTTGGTACCGAGCTCTGATCCAGTAGTCGAGTGTGGTGG
AATTCTGCAGATATCCAGCACAGTGGCGGCCGGTGGAGAACAAGTTTGT
ACAAAAAAGCTGAACGAGAAACGTAAAATGATATAAATATCAATATATT
AAATTAGATTTTGCATAAAAAACAGACTACATAATACTGTAAAACACAA
CATATCCAGTCACTATGAATCAACTACTTAGATGGTATTAGTGACCTGTA
GTCGACCGACAGCCTTCCAAATGTTCTTCGGGTGATGCTGCCAACTTAGT
CGACCGACAGCCTTCCAAATGTTCTTGTCAAACGGAATCGTCGTATCGAG
CCTACTCGCTATTGTCCTCAATGCCGTATTAAATCATAAAAAGAAATAAG
AAAAAGAGGTGCGAGCCTCTTTTTTGTGTGACAAAATAAAAACATCTAC
CTATTCATATACGCTAGTGTCATAGTCCTGAAAATCATCTGGATCAAGAA
CAATTTCACAACTCTTATACTTTTCTCTTACAAGTCGTTCGGCTTCATCTG
GATTTTCAGCCTCTATACTTACTAAACGTGATAAAGTTTCTGTAATTTCTA
CTGTATCGACCTGCAGACTGGCTGTGTATAAGGGAGCCTGACATTTATAT
TCCCCAGAACATCAGGTTAATGGCGTTTTTGATGTCATTTTCGCGGTGGC
TGAGATCAGCCACTTCTTCCCCGATAACGGAGACCGGCACACTGGCCAT
ATCGGTGGTCATCATGCGCCAGCTTTCATCCCCGATATGCACCACCGGGT
AAAGTTCACGGGAGACTTTATCTGACAGCAGACGTGCACTGGCCAGGGG
GATCACCATCCGTCGCCCGGGCGTGTGAATAATATCACTCTGTACATCCA
CAAACAGACGATAACGGCTCTCTCTTTTATAGGTGTAAACCTTAAACTGC
ATTTCACCAGTCCCTGTTCTCGTCAGCAAAAGAGCCGTTCATTTCAATAA
ACCGGGCGACCTCAGCCATCCCTTCCTGATTTTCCGCTTTCCAGCGTTCG
GCACGCAGACGACGGGCTTCATTCTGGATGGTTGTGCTTACCAGACGGG
AGATATTGAGATCATATATGCCTTGAGCAACTGATAGCTGTCGGTGTCAA
CTGTCACTGTAATACGCTGCTTCATAGCACACCTGTTTTTGACATACTTCG
GGTATACATATCAGTATATATTCTTATAGCGCAAAAATCAGCGCGCAAAT
ACGCATACTGTTATCTGGCTTTTAGTAAGCCGGATCCACGCGATTACGCC
CCGCCCTGCCACTCATCGCAGTACTGTTGTAATTCATTAAGCATTCTGCC
GACATGGAAGCCATCACAGACGGCATGATGAACCTGAATCGCCAGCGGC
ATCAGCACCTTGTCGCCTTGCGTATAATATTTGCCCATGGTGAAAACGGG
GGCGAAGAAGTTGTCCATATTGGCCACGTTTAAATCAAAAGTGGTGAAA
CTCACCCAGGGATTGGCTGAGACGAAAAAGATATTCTCAATAAACCCTT
TAGGGAAATAGGCCAGGTTTTCACCGTAACACGCCACATCTTGGGAATA
TATGTGTAGAAACTGCCGGAAATCGTCGTGGTATTCACTCCAGAGCGAT
GAAAACGTTTCAGTTTGCTGATGGAAAACGGTGTAACAAGGGTGAACAG
TATCCCATATCACCAGCTCACCGTCTTTCATTGCCATACGGAATTCCGGA
TGAGCATTCATCAGGCGGGCAAGAATGTGAATAAAGGCCGGATAAAACT
TGTGCTTATTTTTCTTTACGGTCTTTAAAAAGGCCGTAATATCCAGCTGA
ACGGTGTGGTTATAGGTACATTGAGCAACTGACTGAAATGCCTCAAAAT
GTTCTTTACGATGCCATTGGGATATATCAACGGTGGTATATCCAGTGATT
TTTTTCTCCATTTTAGCTTCCTTAGCTCCTGAAAATCTCGATAACTCAAAA
AATACGCCGGGTAGTGATCTTATTTCATTATGGTGAAAGTTGGAACCTCT
TACGTGGCGATCAACGTCTCATTTTCGCCAAAAGTTGGGCCAGGGCTTCC
CGGTATGAACAGGGAGACCAGGATTTATTTATTCTGCGAAGTGATCTTCC
GTCACAGGTATTTATTGGGCGCAAAGTGCGTCGGGTGATGCTGCCAACTT
AGTCGACTACAGGTCAGTAATACCATCTAAGTAGTTGATTCATAGTGACT
GGATATGTTGTGTTTTACAGTATTATGTAGTCTGTTTTTTATGCAAAATCT
AATTTAATATATTGATATTTATATCATTTTACGTTTGTCGTTCAGCTTTCT
TGTACAAAGTGGTTGATCTGTGAATTCTTGGAGTGGTGAATCCGTTAGCG
AGGTGCCGCGCTGCTTCATCCCCGTGGCCCGTTGCTCGCGTTTGCTGGCG
GTGTCCCCGGAAGAAATATATTTGCATGTCTTTAGTTCTATGATGACACA
AACCCCGCCCAGCGTCTTGTCATTGGCGAATTCGAACACGCAGATGCAG
TCGGGGCGGCGCGGTCCGAGGTCCACTTCGCATATTAAGGTGACGCGTG
TGGCCTCGAACACCGAGCGACCCTGCAGCGACCCGCTTAACAGCGTCAA
GAGCGTGCCGCAGATCAGCTTGATATGAAAAAGCCTGAACTCACGGCGA
CGTCTGTCGAGAAGTTTCTGATCGAAAAGTTCGACAGCGTCTCCGACCTG
ATGCAGCTCTCGGAGGGCGAAGAATCTCGTGCTTTGAGCTTCGATGTAG
GAGGGCGTGGATATGTCCTGCGGGTAAATAGCTGGGCCGATGGTTTCTA
CAAAGATCGTTATGTTTATCGGCACTTTGCATCGGCCGCGCTCCCGATTC
CGGAAGTGCTTGACATTGGGGAATTCAGCGAGAGCCTGACCTATTGCAT
CTCCCGCCGTGCACAGGGTGTCACGTTGCAAGACCTGCCTGAAACCGAA
CTGCCCGCTGTTCTGCAGCCGGTCGCGGAGGCCATGGATGCGATCGCTG
CGGCCGATCTTAGCCAGACGAGCGGGTTCGGCCCATTCGGACCGCAAGG
AATCGGTGAATAGAGTACATGGCGTGATTTCATATGCGCGATTGCTGATC
CCGATGTGTATCACTGGGAAACTGTGATGGACGACACCGTCAGTGCGTC
CGTCGCGCAGGCTCTCGATGAGCTGATGCTTTGGGCCGAGGACTGCCCC
GAAGTCGGGGCACCTCGTGCACGCGGATTTCGGCTCCAACAATGTCCTG
ACGGACAATGGCCGCATAACAGCGGTCATTGACTGGAGCGAGGCGATGT
TCGGGGATTCCCAATACGAGGTCGCCAACATCTTCTTCTGGAGGCCGTGG
TTGGCTTGTATGGAGCAGCAGACGCGCTACTTCGAGCGGAGGCATCCGG
AGCTTGCAGGATCGCCGCGGCTCCGGGCGTATATGCTGCGCATTGGTCTT
GACCAAGTCTATCAGAGCTTGGTTGACGGCAATTCGATGATGCAGCTTG
GGCGCAGGGTCGATGCGACGCAATCGTCCGATCCGGAGCCGGGACTGTC
GGGCGTACACAAATCGCCCGCAGAAGCGCGGCCGTCTGGACCGATGGCT
GTGTAGAAGTACTCGCCGATAGTGGAAACCGACGCCGCAGCACTCGTCC
GGATCGGGAGATGGGGGAGGCTAACTGAATCGATAAAATAAAAGATTTT
ATTTAGTCTCCAGAAAAAGGGGGGAATGAAAGACCCCACCTGTAGGTTT
GGCAAGGTAGCTTAAGTAACGCCATTTTTGCAAGGCATGGAAAAATACAT
AACTGAGAATAGAGAAGTTCAGATGAAGGTAGGAGATCCGTGAGCCCAC
AACCGCTCACTCGGGGCGCCAGTCGTCCGATTGACTGAGTCGCCCGGGT
ACCGGTGTATCCAATAAACGCTCTTGCAGTTGCATGCGAGTTGTGGTCTC
GCTGTTGCTTGGAAGGGTCTCCTCTGAGTGATTGACTACCCGTCAGCGGG
GGTCTTTGACATGCAGCATGTATGAAAATTAATTTGGTTTTTTTTCTTAAG
TATTTACATTAAATGGCCATAGTTGCATTAATGAATCGGGCAACGCGCGG
GGAGAGGCGGTTTGCGTATTGGGCGCTCTTCCGCTTCCTCGGTCACTGAG
TCGCTGCGCTCGGTCGTTCGGCTGCGGCGAGCGGTATCAGCTGACTCAAA
GGGGGTAATACGGTTATCGACAGAATCAGGGGATAACGCAGGAAAGAA
CATGTGAGCAAAAGGCCAGCAAAAGGCCAGGAACCGTAAAAAGGCCGC
GTTGCTGGGGTTTTTCCATAGGCTCCGCCCCCCTGACGAGCATCACAAAA
ATCGACGCTCAAGTCAGAGGTGGGGAAACCCGACAGGACTATAAAGATA
CCAGGCGTTTCCCCCTGGAAGCTCCCTCGTGCGCTCTCCTGTTCCGACCC
TGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCG
CTCCAAGCTGGGCTGTGTGCACGAACGCCCCGTTCAGCCCGACCGCTGG
GCCTTATCCGGTAACTATCGTCTTGAGTCCAACCCGGTAAGACACGACTT
ATCGCCACTGGCAGCAGCCACTGGTAACAGGATTAGCAGAGCGAGGTAT
GTAGGCGGTGCTACAGAGTTCTTGAAGTGGTGGCCTAACTACGGGTACA
CTAGAAGAACAGTATTTGGTATCTGCGCTCTGCTGAAGCCAGTTACGTTC
GGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGG
ATCTCAAGAAGATCCTTTGATCTTTTCTACGGGGTCTGACGCTCAGTGGA
ACGAAAACTCACGTTAAGGGATTTTGGTCATGAGATTATCAAAAAGGAT
CTTCACCTAGATCCTTTTGCGGCCGCAAATCAATCTAAAGTATATATGAG
TAAACTTGGTCTGACAGTTACCAATGCTTAATCAGTGAGGCACCTATCTC
AGCGATCTGTGTATTTCGTTCATCCATAGTTGCCTGACTCCCCGTCGTGTA
GATAACTACGATACGGGAGGGCTTACCATCTGGCCCCAGTGCTGCAATG
ATACCGCGAGACCCACGCTCACCGGCTCCAGATTTATCAGCAATAAACC
AGCCAGCCGGAAGGGCCGAGCGCAGAAGTGGTCCTGCAACTTTATGCGC
CTCCATCCAGTCTATTAATTGTTGCCGGGAAGCTAGAGTAAGTAGTTCGC
CAGTTAATAGTTTGCGCAACGTTGTTGCCATTGCTACAGGCATGGTGGTG
TCACGCTCGTCGTTTGGTATGGCTTCATTCAGCTCCGGTTCCCAACGATC
AAGGCGAGTTACATGATGCCCCATGTTGTGCAAAAAAGCGGTTAGCTCC
TTCGGTCCTGCGATCGTTGTCAGAAGTAAGTTGGGCGCAGTGTTATCACT
CATGGTTATGGCAGCAGTGGATAATTCTCTTACTGTCATGCCATCCGTAA
GATGCTTTTCTGTGACTGGTGAGTACTCAACCAAGTCATTCTGAGAATAG
TGTATGCGGCGACGGAGTTGCTCTTGCCCGGCGTCAATACGGGATAATA
CCGCGCCACATAGGAGAACTTTAAAAGTGCTCATCATTGGAAAACGTTC
TTCGGGGCGAAAACTCTCAAGGATCTTACCGCTGTTGAGATCCAGTTCGA
TGTAACCCACTCGTGCACCCAACTGATCTTCAGCATCTTTTACTTTCACC
AGCGTTTCTGGGTGAGCAAAAACAGGAAGGCAAAATGCCGCAAAAAAG
GGAATAAGGGCGAGACGGAAATGTTGAATACTCATACTCTTCCTTTTTCA
ATATTATTGAAGCATTTATCAGGGTTATTGTCTCATGAGCGGATACATAT
TTGAATGTATTTAGAAAAATAAACAAATAGGGGTTCCGCGCAGATTTCC
CCGAAAAGTGCCAC

1. A regulated polymerase III expression system, comprising
(a) a first nucleic acid segment comprising a regulated promoter operably linked to a first element encoding a transcription factor; and
(b) a second nucleic acid segment comprising a recombinant polymerase III promoter regulated by the transcription factor,
wherein the transcription factor increases transcription from the recombinant polymerase III promoter.

2. The expression system of claim 1, wherein binding of the transcription factor to (i) the polymerase III promoter or to (ii) at least one binding site operably linked to the polymerase III promoter increases transcription from the recombinant polymerase III promoter.

3. The expression system of claim 1, wherein the first and second nucleic acid segments reside in the same nucleic acid.

4. A nucleic acid comprising the first and second nucleic acid segments of claim 1.

5. The nucleic acid of the preceding claim comprising the nucleic acid sequence as set forth in SEQ ID NO: 1.

6. A nucleic acid comprising the nucleic acid sequence as set forth in SEQ ID NO: 1.

7. A nucleic acid comprising the nucleic acid sequence as set forth in SEQ ID NO: 2.

8. A cell comprising the regulated polymerase III expression system of claim 1.

9. A non human organism comprising the cell of claim 8.

10. A non human organism comprising the regulated polymerase III expression system of claim 1.

11. The expression system of the claim 1, wherein the regulated promoter is an inducible promoter.

12. The expression system of claim 11, wherein transcription from the inducible promoter is increased in the presence of an ecdysone, an ecdysone-analog or an ecdysone mimic.

13-14. (canceled)

15. The expression system of claim 1, wherein transcription from the regulated promoter is developmentally regulated.

16. The expression system of the claim 1, wherein transcription from the regulated promoter is tissue specific.

17. The expression system of the claim 1, wherein transcription from the regulated promoter is temporally regulated.

18. The expression system of the claim 1, wherein transcription from the regulated promoter is cell-cycle regulated.

19. The expression system of claim 1, wherein the regulated promoter comprises or is operably linked to at least one ecdysone response element.

20. The expression system of claim 1, wherein the transcription factor comprises a DNA-binding domain and a transactivating domain.

21. The expression system of the preceding claim, wherein the DNA-binding domain is a GAL4 DNA-binding domain.

22. The expression system of claim 1, wherein the DNA-binding domain does not comprise a tet DNA-binding domain.

23. The expression system of claim 20, wherein the transactivating domain is an Oct-1 or an Oct-2 domain.

24. The expression system of claim 20, wherein the transactivating domain is an Oct-2^{Q}(Q→A) domain.

25. The expression system of claim 20, wherein the transcription factor binds to at least one binding site operably linked to the polymerase III promoter.

26. The expression system of claim 1, wherein the transcription factor does not bind an inducer.

27. The expression system of the preceding claim, wherein the inducer is tetracycline or doxycycline.

28. The expression system of claim 1, wherein expression of the transcription factor is dependent on the presence of an inducer.

29. The expression system of claim 1, wherein transcription from the recombinant polymerase III promoter is dependent on the presence of an inducer.

30. The expression system of the preceding claim, wherein the transcription factor regulates transcription from the recombinant RNA polymerase III promoter by binding to (i) at least one binding site operably linked to said promoter; or (ii) to said promoter.

31. The expression system of the preceding claim, wherein binding of the transcription factor to the recombinant RNA polymerase promoter by or to a binding site operably linked to said promoter increases transcription from said promoter.

32. The method of claim 29, wherein binding affinity of the transcription factor for (i) the polymerase III promoter or for (ii) the binding site operably linked to said promoter is substantially the same in the presence or absence of the inducer.

33. The expression system of claim 1, wherein the polymerase III promoter is a mammalian promoter.

34. The expression system of claim 1, wherein the polymerase III promoter element comprises a U6 promoter or an H1 promoter.

35-36. (canceled)

37. The expression system of claim 1, wherein the second nucleic acid segment comprises at least one binding site for the transcription factor operably linked to the recombinant polymerase III promoter.

38-42. (canceled)

43. The expression system of claim 1, wherein the regulated promoter is further operably linked to a second element.

44. The expression system of the preceding claim, wherein the second element encodes a reporter protein, a selectable marker or an enzyme.

45. The expression system of the preceding claim, wherein the reporter protein comprises a fluorescent protein.

46. The expression system of the preceding claim, wherein the fluorescent protein comprises a GFP protein.

47. The expression system of the preceding claim, wherein the selectable marker comprises a cell surface receptor or a drug-resistance marker.

48-49. (canceled)

50. The expression system of claim 1, further comprising a sequence of a transgene operably linked to the recombinant polymerase III promoter.

51. The expression system of claim 50, wherein the transgene encodes a non-coding RNA.

52. The expression system of claim 51, wherein the non-coding RNA comprises an siRNA.

53. The expression system of claim 51, wherein the transgene comprises a hairpin RNA.

54. The expression system of claim 51, wherein the transgene comprises a ribozyme.

55. (canceled)

56. The expression system of claim 51, wherein the non-coding RNA inhibits the expression of an essential gene.

57. The expression system of claim 1, further comprising a cloning site downstream of the polymerase III promoter.

58. The expression system of the preceding claim, wherein the cloning site comprises a restriction enzyme recognition site or a ccdB sequence.

59. (canceled)

60. The expression system of claim 1, comprising at least one nucleic acid segment encoding a regulatory protein which promotes transcription from the regulated promoter.

61. The expression system of the preceding claim, comprising a nucleic acid segment encoding two regulatory proteins which promote transcription from the regulated promoter.

62. (canceled)

63. The expression system of claim 60, wherein the regulatory protein binds to an inducer.

64. The expression system of the preceding claim, wherein binding of the regulatory protein to the inducer promotes transcription from the regulated promoter.

65. The expression system of the claim 63, wherein binding of the regulatory protein to the inducer promotes binding of the regulatory protein to a response element.

66. The expression system of claim 60, wherein the regulatory protein binds to the regulated promoter or to a response element operably linked to the regulated promoter.

67. The expression system of the preceding claim, wherein binding of the regulatory protein to the regulated promoter or to a response element operably linked to the regulated promoter promotes transcription from the regulated promoter.

68. The expression system of claim 60, wherein the regulatory protein does not bind to the polymerase III promoter.

69. The expression system of claim 60, wherein the regulatory protein comprises a DNA binding domain.

70. The expression system of the preceding claim, wherein the DNA-binding domain of the regulatory protein comprises a tet repressor DNA binding domain, an RxR DNA binding domain or a nuclear hormone receptor DNA binding domain.

71. The expression system of claim 60, wherein the regulatory protein promotes transcription from the regulated promoter upon binding to an inducer.

72. The expression system of the preceding claim, wherein the inducer is tetracycline, ecdysone hormone, or an agonist thereof.

73. The expression system of claim 60, wherein the protein is a nuclear receptor or a transcription factor.

74. The expression system of the preceding claim, wherein the protein comprises a VgEcR or an RXR protein.

75. A method of reducing gene expression of a gene in a cell, the method comprising
(a) providing a cell comprising
(i) a regulated promoter operably linked to a first element encoding a transcription factor; and
(ii) a recombinant polymerase III promoter regulated by the transcription factor and operably linked to a coding sequence for an RNA molecule, wherein expression of the RNA molecule reduces expression of the gene; and
(b) contacting the cell with an inducer, wherein the inducer promotes transcription of the RNA molecule from the recombinant polymerase III promoter,
thereby reducing expression of the gene in the cell.

76. A method of determining the effects of reducing gene expression of a gene in a cell, the method comprising
(a) providing a cell comprising
(i) a regulated promoter operably linked to a first element encoding a transcription factor; and
(ii) a recombinant polymerase III promoter regulated by the transcription factor and operably linked to a coding sequence for an RNA molecule, wherein expression of the RNA molecule reduces expression of the gene;
(b) subjecting the cell to a condition which promotes transcription of the RNA molecule from the recombinant polymerase III promoter; and
(c) determining the phenotype of the cell;
thereby determining the effects of reducing expression of the gene.

77. A method of determining the effects of reducing gene expression of a gene in an organism, the method comprising
(a) providing an organism wherein at least a cell in the organism comprises
(i) a regulated promoter operably linked to a first element encoding a transcription factor; and
(ii) a recombinant polymerase III promoter regulated by the transcription factor and operably linked to a coding sequence for an RNA molecule, wherein expression of the RNA molecule reduces expression of the gene;
(b) subjecting the organism to conditions which promote transcription of the RNA molecule from the recombinant polymerase III promoter in at least one cell; and
(c) determining the phenotype of at least one cell in the organism;
thereby determining the effects of silencing expression of a gene in an organism.

78-96. (canceled)


SEQUENCE LISTING (2 sequences)

SEQ ID NO: 1 - DNA, 327 bp, Artificial Sequence
  Gal-4 Binding Site/U6 Promoter Cassette
gcggccgccgagctcggtaccccgacggagtactgtcctccgacggagtactgtcctccgacggagtactgtcctctgacgagtactgtcctccgacggggatcctctagagtcatcgagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtttttttgcggccgc

SEQ ID NO: 2 - DNA, 9772 bp, Artificial Sequence
  Plasmid pEind-RNAi
ctgcagcctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccggaaggcaagctggccagcaacttatctgtgtctgtccgattgtctagtgtctatgactgattttatgcgcctgcgtcggtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagacttgtaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggccggatctcggccgcatattaagtgcattgttctcgataccgctaagtgcattgttctcgttagctcgatggacaagtgcattgttctcttgctgaaagctcgatggacaagtgcattgttctcttgctgaaagctcgatggacaagtgcattgttctcttgctgaaagctcagtacccgggagtaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatactttcaacaagttaccgagaaagaagaactcacacacagctagcgtttaaaccgaattctgcagtcgacggtaccgcgatgaagctactgtcttctatcgaacaagcatgcgatatttgccgacttaaaaagctcaagtgctccaaagaaaaaccgaagtgtgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacactgccaggctctagaccaaatctattcgccctacctgccgctaccgccggagctcttctgacctccgcaccaaatctattcgccctacctgccgctaccgccggagctcttctgacctccgcaccaaatctattcgccctacctgccgctaccgccggagctcttctgacctccgcaccaaatctattcgccctacctgccgctaccgccggagctcttctgacctccgcaccatagtcttcggatccgcccctctccctcccccccccctaacgttactggccgaagccgcttggaataaggccggngtgcgtttgtctatatgttattttccaccatattgccgtcttttggcaatgtgagggcccggaaacctggccctgtcttcttgacgagcattcctaggggtctttcccctctcgccaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatctcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacttaagcttggtaccgagctctgatccactagtccagtgtggtggaattctgcagatatccagcacagtggcggccgctcgagaacaagtttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtggttgatctctgaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcagcttgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccgggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtaggagatccctgagcccacaacccctcactcggggcgccagtcctccgattgactgagtcgcccgggtacccgtgtatccaataaaccctcttgcagttgcatccgacttgtggtctcgctgttccttggaagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac